(19) United States
(12) Patent Application Publication
(10) Pub. No.: US 2022/0104495 A1
(43) Pub. Date: Apr. 7, 2022

(54) THEOPHYLLINE DERIVATIVES WITH NEMATOCIDAL ACTIVITY, THEIR AGRONOMIC COMPOSITIONS AND RELATIVE USE

(71) Applicant: ISAGRO S.p.A., Milan (IT)

(72) Inventors: Marilena GUSMEROLI, Monza (MB) (IT); Serena CARIELLO, Novara (IT); Giuseppe D?ORAZIO, Bollate (MI) (IT); Paolo BELLANDI, Carcare (SV) (IT); Chiara SARGIOTTO, Galliate (NO) (IT); Daniele BIANCHI, Bellinzago Novarese (NO) (IT); Jessica VILLATA, Cameri (NO) (IT); Riccardo LIGUORI, Monza (MB) (IT)

(21) Appl. No.: 17/424,810

(22) PCT Filed: Jan. 29, 2020
(86) PCT No.: PCT/IB2020/050685

§ 371 Date: Jul. 21, 2021

(30) Foreign Application Priority Data
Jan. 30, 2019 (IT) 102019000001339

(51) Int. Cl. A01N 43/90; C07D 473/08; A01P 5/00

(52) U.S. Cl. CPC A01N 43/90; A01P 5/00; C07D 473/08

(57) ABSTRACT

The present invention related to theophylline derivatives having general formula (I) agronomic compositions containing said compounds having formula (I) and analogous compounds having formula (XVI) and their use for the control of nematodes in agricultural crops. These compounds advantageously prove to be both effective against parasites and free of phytotoxicity.

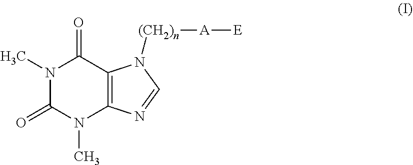

The present invention relates to theophylline derivatives having general formula (I)

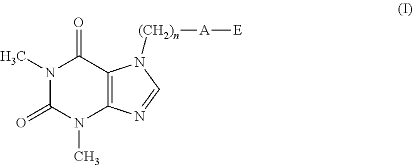

The present invention also relates to agronomic compositions containing said compounds having formula (I) and their use for the control of nematodes in agricultural crops.

### STATE OF THE ART

Parasitic plant nematodes represent a serious threat to horticultural crops. Nematodes, small worm-like organisms present in the soil, are in fact able to settle in the roots of plants and live at their expense, thus slowing down their development and growth due to the insufficient supply of nutrients.

Various techniques are currently used for fighting nematodes, among which solarization of the soil, treatment with biological and/or chemical products or the use of cultivars resistant to nematodes, can be mentioned. With respect to the chemical control, the compounds belonging to the class of carbamates and organophosphorus compounds, mainly used as chemical nematocides, are ultimately limited or even abandoned due to problems of toxicity or environmental impact.

The need is therefore felt for identifying new molecules having a reduced environmental impact that can effectively restrain the attack of nematodes on crops.

Italian patent IT1368843 indicates that caffeine, a molecule of natural origin in which position 7 of the theophylline ring is substituted by an additional methyl group, can be validly used against some species of nematodes that attack horticultural crops.

The Applicant however has verified that this compound is unsatisfactory from the point of view of nematocidal activity, as it cannot effectively limit the attack of the parasite and reduce the formation of galls on the root system of the plant. Furthermore, caffeine is phytotoxic with respect to important agricultural crops, showing a significant necrosis of the leaves and stem, at the doses that allow a good nematocidal activity to be obtained.

To the knowledge of the Applicant, caffeine or theophylline derivatives substituted in position 7 with a nematocidal activity are not known in literature.

### DESCRIPTION

The Applicant has now surprisingly found that by introducing suitable substituents in position 7 of the theophylline ring, products are obtained having a considerable nematocidal activity with respect to numerous phytoparasitic nematodes.

At the same time, these products, at effective doses, exhibit a low or zero phytotoxicity for crops of agricultural interest and can therefore be used as nematocides.

A first object of the present invention therefore relates to a compound having formula (I):

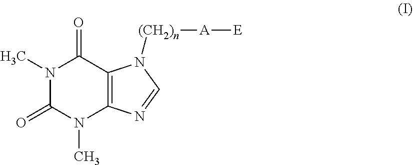

wherein:
n represents an integer ranging from 0 to 3;
A represents a group selected from: —S—, —S(═O)—, —S(═O)₂—, —C(═Y)—O—, —C(═Y)—NR₁—S(O)₂—;
Y represents an oxygen or sulfur atom;
R₁ represents a hydrogen atom, a C₁-C₆ alkyl group, a C₁-C₆ haloalkyl group, a C₂-C₆ alkenyl group, a C₂-C₆ haloalkenyl group, a C₃-C₈ cycloalkyl group, a C₄-C₉ cycloalkylalkyl group, a C(═Y)R₂ group, a C(═O)OR₂ group, a C(═O)NR₂R₃ group, an S(═O)ᵣR₂ group, an S(═O)₂NR₂R₃ group;
R₂ and R₃, the same as or different from each other, represent a hydrogen atom, a C₁-C₆ alkyl group, a C₁-C₆ haloalkyl group, a C₂-C₆ alkenyl group, a C₂-C₆ haloalkenyl group, a C₃-C₈ cycloalkyl group, a C₃-C₈ halocycloalkyl group, an aryl group, a benzyl group;
r represents an integer ranging from 0 to 2;
E represents a —(CH₂)ₘ—CX═CF₂ group or a group selected from a C₁-C₆ alkyl group, a C₁-C₆ haloalkyl group, a C₂-C₆ alkenyl group, a C₂-C₆ haloalkenyl group, a C₂-C₆ alkynyl group and a C₂-C₆ haloalkynyl group, optionally substituted by a C₁-C₆ alkoxyl group;
m represents an integer ranging from 1 to 6;
X represents a hydrogen or fluorine atom;

with the proviso that when A represents a group selected from: —S—, —S(═O)—, —S(═O)₂—, —C(═Y)—O—, then E represents the group —(CH₂)ₘ—CX═CF₂.

In the present description, when a numerical range is indicated, the extremes are also meant to be included in the same.

Examples of halogen are: fluorine, chlorine, bromine, iodine.

Examples of C₁-C₆ alkyl, linear or branched, are methyl, ethyl, n-propyl, isopropyl, n-butyl, isobutyl, sec-butyl, tert-butyl, n-pentyl, 3-methylbutyl, n-hexyl, 3,3-dimethylbutyl.

Examples of C₁-C₆ haloalkyl are fluoromethyl, difluoromethyl, trifluoromethyl, chloromethyl, dichloromethyl, 2,2,2-trifluoroethyl, 1,1,2,2-tetrafluoroethyl, pentafluoroethyl, heptafluoropropyl, 4,4,4-trichloro-butyl, 4,4-difluoropentyl, 5,5-difluorohexyl.

Examples of C₂-C₆ alkenyl, linear or branched, are vinyl, allyl, 3-butenyl, 4-pentyl.

Examples of C₂-C₆ haloalkenyl are 1-(1,1,2-trifluoro)-butenyl, 1-(2,2-difluoro)-butenyl.

Examples of C₃-C₈ cycloalkyl are cyclopropyl, cyclobutyl, cyclopentyl, cyclohexyl.

Examples of C₃-C₈ halocycloalkyl are 2,2-dichloro-cyclopropyl, 2,2-difluorocyclopropyl, 2,2,3,3-tetrafluoro-cyclobutyl, 3,3-difluorocyclopentyl, 2-fluorocyclohexyl.

Examples of C₄-C₉ cycloalkylalkyls are cyclopropylmethyl, 2-cyclopentylethyl, 2-cyclohexylpropyl.

Examples of aryl are phenyl, 2-chloro-5-methoxy-phenyl, 2-chloro-6-fluoro-phenyl, 2-chloro-phenyl, 2,4,6-trifluoro-phenyl.

Examples of C₂-C₆ alkynyls are ethynyl, 1-propynyl, 2-propynyl, 2-pentinyl.

Examples of C₂-C₆ haloalkyls are 3-bromopropyne, 4-bromopentyne, 3-chlorobutyne.

The following also fall within the spirit of the present invention:

a) all possible geometric isomers of the compounds having general formula (I);

b) the salts of the compounds having formula (I) obtained by the addition of inorganic or organic acids;

c) any possible hydrated, solvated and polymorphic forms of the compounds having formula (I);

d) all possible optical isomers and their mixtures, including racemic mixtures of the compounds having formula (I).

Preferred compounds having formula (I-A) are compounds having general formula (I) wherein:
n is an integer ranging from 0 to 2,
R₁ represents a hydrogen atom or a C₁-C₆ alkyl group,
E represents a —(CH₂)ₘ—CX═CF₂ group or a group selected from a C₁-C₆ alkyl group and a C₁-C₆ haloalkyl group, optionally substituted by a C₁-C₆ alkoxyl group,
m represents an integer ranging from 1 to 5.

Preferred compounds having formula (I-B) are compounds having general formula (I) wherein:
n is an integer selected from 1 and 2;
A represents a group selected from: —S(═O)—, —S(═O)₂—, —C(═O)—O—, —C(═O)—NR₁—S(O)₂—;
R₁ represents a hydrogen atom or a C₁-C₆ alkyl group;
E represents a —(CH₂)ₘ—CX═CF₂ group or a C₁-C₆ alkyl group optionally substituted by a C₁-C₆ alkoxyl group,
m represents an integer ranging from 1 to 4.

Preferred compounds having formula (I-C) are compounds having general formula (I) wherein:
n is an integer ranging from 1 to 2;
A represents a group selected from: —S(═O)—, —S(═O)₂—, —C(═O)—O—, —C(═O)—NR₁—S(O)₂—;
R₁ represents a hydrogen atom or a C₁-C₆ alkyl group;
E represents a —(CH₂)ₘ—CX═CF₂ group;
m represents an integer ranging from 1 to 4.

Preferred compounds having formula (I-D) are compounds having general formula (I) wherein:
E represents a —(CH₂)ₘ—CX═CF₂ group; and
n is selected from 1 and 2.

Preferred compounds having formula (I-E) are compounds having general formula (I) wherein:
E represents a —(CH₂)ₘ—CX═CF₂ group;
m is 2;
n is selected from 1 and 2;
A represents a group selected from: —S(═O)—, —S(═O)₂—, —C(═O)—O—, —C(═O)—NR₁—S(O)₂—.

Specific examples of compounds having general formula (I), interesting for their nematocidal activity, are compounds wherein n, A and E have the meanings indicated in table 1:

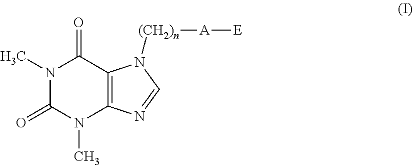

TABLE 1

| Comp. | | | |
|---|---|---|---|
| N^{r} | A | n | E |
| 1 | —C(O)O— | 0 | —(CH₂)₂—CF═CF₂ |
| 2 | —C(O)O— | 0 | —(CH₂)₂—CH═CF₂ |
| 3 | —C(O)O— | 0 | —(CH₂)₄—CF═CF₂ |
| 4 | —C(O)O— | 2 | —(CH₂)₂—CF═CF₂ |
| 5 | —S— | 0 | —(CH₂)₂—CF═CF₂ |
| 6 | —S— | 0 | —(CH₂)₂—CH═CF₂ |
| 7 | —S— | 2 | —(CH₂)₂—CF═CF₂ |
| 8 | —S(O)— | 2 | —(CH₂)₂—CF═CF₂ |
| 9 | —S(O)— | 0 | —(CH₂)₂—CF═CF₂ |
| 10 | —S(O)— | 0 | —(CH₂)₂—CH═CF₂ |
| 11 | —S(O)₂— | 2 | —(CH₂)₂—CF═CF₂ |
| 12 | —S(O)— | 0 | —(CH₂)₄—CH═CF₂ |
| 13 | —S(O)₂— | 0 | —(CH₂)₂—CF═CF₂ |
| 14 | —S(O)₂— | 0 | —(CH₂)₂—CH═CF₂ |
| 15 | —S(O)₂— | 0 | —(CH₂)₄—CF═CF₂ |
| 16 | —S(O)₂— | 0 | —(CH₂)₄—CH═CF₂ |
| 17 | —C(S)O— | 0 | —(CH₂)₂—CF═CF₂ |
| 18 | —C(S)O— | 0 | —(CH₂)₂—CH═CF₂ |
| 19 | —C(S)O— | 0 | —(CH₂)₄—CF═CF₂ |
| 20 | —C(S)O— | 0 | —(CH₂)₄—CH═CF₂ |
| 21 | —C(O)O— | 1 | —(CH₂)₂—CF═CF₂ |
| 22 | —C(O)O— | 1 | —(CH₂)₂—CH═CF₂ |
| 23 | —C(O)O— | 1 | —(CH₂)₄—CF═CF₂ |
| 24 | —C(O)O— | 1 | —(CH₂)₄—CH═CF₂ |
| 25 | —S— | 1 | —(CH₂)₂—CF═CF₂ |
| 26 | —S— | 1 | —(CH₂)₂—CH═CF₂ |
| 27 | —S— | 1 | —(CH₂)₄—CF═CF₂ |
| 28 | —S— | 1 | —(CH₂)₄—CH═CF₂ |
| 29 | —S(O)— | 1 | —(CH₂)₂—CF═CF₂ |
| 30 | —S(O)— | 1 | —(CH₂)₂—CH═CF₂ |
| 31 | —S(O)— | 1 | —(CH₂)₄—CF═CF₂ |
| 32 | —S(O)— | 1 | —(CH₂)₄—CH═CF₂ |
| 33 | —S(O)₂— | 1 | —(CH₂)₂—CF═CF₂ |
| 34 | —S(O)₂— | 1 | —(CH₂)₂—CH═CF₂ |
| 35 | —S(O)₂— | 1 | —(CH₂)₄—CF═CF₂ |
| 36 | —S(O)₂— | 1 | —(CH₂)₄—CH═CF₂ |
| 37 | —C(S)O— | 1 | —(CH₂)₂—CF═CF₂ |
| 38 | —C(S)O— | 1 | —(CH₂)₂—CH═CF₂ |
| 39 | —C(S)O— | 1 | —(CH₂)₄—CF═CF₂ |
| 40 | —C(S)O— | 1 | —(CH₂)₄—CH═CF₂ |
| 41 | —C(O)—NH—S(O)₂— | 0 | —(CH₂)₂—CF═CF₂ |
| 42 | —C(O)—NH—S(O)₂— | 0 | —(CH₂)₂—CH═CF₂ |
| 43 | —C(O)—NH—S(O)₂— | 0 | 2-propyl |
| 44 | —C(O)—NH—S(O)₂— | 0 | 2-methyl-1-propyl |
| 45 | —C(O)—NH—S(O)₂— | 0 | 2-methoxy-1-ethyl |
| 46 | —C(O)—NH—S(O)₂— | 0 | 2-trifluoro-1-ethyl |
| 47 | —C(O)—NH—S(O)₂— | 0 | 3-bromo-1-butyl |
| 48 | —C(O)—NH—S(O)₂— | 1 | 2-propyl |
| 49 | —C(O)—NH—S(O)₂— | 1 | 2-methyl-1-propyl |
| 50 | —C(O)—NH—S(O)₂— | 1 | 2-methoxy-1-ethyl |
| 51 | —C(O)—NH—S(O)₂— | 1 | 2-trifluoro-1-ethyl |
| 52 | —C(O)—NH—S(O)₂— | 1 | 3-bromo-1-butyl |
| 53 | —C(O(—N(Me(—S(O)₂— | 0 | —(CH₂)₂—CF═CF₂ |
| 54 | —C(O(—N(Me(—S(O)₂— | 0 | —(CH₂)₂—CH═CF₂ |
| 55 | —C(O(—N(Me(—S(O)₂— | 0 | 2-propyl |
| 56 | —C(O(—N(Me(—S(O)₂— | 0 | 2-trifluoro-1-ethyl |
| 57 | —C(O(—N(Me(—S(O)₂— | 0 | 2-methyl-1-propyl |
| 58 | —C(O(—N(Me(—S(O)₂— | 0 | 2-methoxy-1-ethyl |
| 59 | —C(O(—N(Me(—S(O)₂— | 0 | 3-bromo-1-butyl |
| 60 | —C(O(—N(Me(—S(O)₂— | 1 | —(CH₂)₂—CF═CF₂ |
| 61 | —C(O(—N(Me(—S(O)₂— | 1 | —(CH₂)₂—CH═CF₂ |
| 62 | —C(O(—N(Me(—S(O)₂— | 1 | 2-propyl |
| 63 | —C(O(—N(Me(—S(O)₂— | 1 | 2-trifluoro-1-ethyl |
| 64 | —C(O(—N(Me(—S(O)₂— | 1 | 2-methyl-1-propyl |
| 65 | —C(O(—N(Me(—S(O)₂— | 1 | 2-methoxy-1-ethyl |
| 66 | —C(O(—N(Me(—S(O)₂— | 1 | 3-bromo-1-butyl |
| 67 | —C(O)—NH—S(O)₂— | 1 | —(CH₂)₂—CF═CF₂ |
| 68 | —C(O)—NH—S(O)₂— | 1 | —(CH₂)₂—CH═CF₂ |
| 69 | —C(O)—NH—S(O)₂— | 2 | 2-propyl |
| 70 | —C(O)—NH—S(O)₂— | 2 | 2-trifluoro-1-ethyl |
| 71 | —C(O)—NH—S(O)₂— | 2 | 2-methyl-1-propyl |
| 72 | —C(O)—NH—S(O)₂— | 2 | 2-methoxy-1-ethyl |
| 73 | —C(O)—NH—S(O)₂— | 2 | 3-bromo-1-butyl |
| 74 | —C(O)—NH—S(O)₂— | 2 | —(CH₂)₂—CF═CF₂ |
| 75 | —C(O)—NH—S(O)₂— | 2 | —(CH₂)₂—CH═CF₂ |
| 76 | —C(S)—NH—S(O)₂— | 1 | —(CH₂)₂—CF═CF₂ |
| 77 | —C(S)—NH—S(O)₂— | 1 | —(CH₂)₂—CH═CF₂ |
| 78 | —C(S)—NH—S(O)₂— | 1 | 2-propyl |
| 79 | —C(S)—NH—S(O)₂— | 1 | 2-trifluoro-1-ethyl |
| 80 | —C(S)—NH—S(O)₂— | 1 | 2-methyl-1-propyl |
| 81 | —C(S)—NH—S(O)₂— | 1 | 2-methoxy-1-ethyl |
| 82 | —C(S)—N(Me)—S(O)₂— | 1 | —(CH₂)₂—CF═CF₂ |
| 83 | —C(S)—N(Me)—S(O)₂— | 1 | —(CH₂)₂—CH═CF₂ |
| 84 | —C(S)—N(Me)—S(O)₂— | 1 | 2-propyl |
| 85 | —C(S)—N(Me)—S(O)₂— | 1 | 2-trifluoro-1-ethyl |
| 86 | —C(S)—N(Me)—S(O)₂— | 1 | 2-methyl-1-propyl |
| 87 | —C(S)—N(Me)—S(O)₂— | 1 | 2-methoxy-1-ethyl |
| 88 | —C(S)—NH—S(O)₂— | 2 | —(CH₂)₂—CF═CF₂ |
| 89 | —C(S)—NH—S(O)₂— | 2 | —(CH₂)₂—CH═CF₂ |
| 90 | —C(S)—NH—S(O)₂— | 2 | 2-propyl |
| 91 | —C(S)—NH—S(O)₂— | 2 | 2-trifluoro-1-ethyl |
| 92 | —C(S)—NH—S(O)₂— | 2 | 2-methyl-1-propyl |
| 93 | —C(S)—NH—S(O)₂— | 2 | 2-methoxy-1-ethyl |
| 94 | —C(S)—N(Me)—S(O)₂— | 2 | —(CH₂)₂—CF═CF₂ |
| 95 | —C(S)—N(Me)—S(O)₂— | 2 | —(CH₂)₂—CH═CF₂ |
| 96 | —C(S)—N(Me)—S(O)₂— | 2 | 2-propyl |
| 97 | —C(S)—N(Me)—S(O)₂— | 2 | 2-trifluoro-1-ethyl |

Particularly preferred are compounds having general formula (I) wherein n, A and E have the meanings indicated in table 2:

TABLE 2

| Comp. | | | |
|---|---|---|---|
| N^{r} | A | n | E |
| 5 | —S— | 0 | —(CH₂)₂—CF═CF₂ |
| 7 | —S— | 2 | —(CH₂)₂—CF═CF₂ |
| 8 | —S(O)— | 2 | —(CH₂)₂—CF═CF₂ |
| 9 | —S(O)— | 0 | —(CH₂)₂—CF═CF₂ |
| 10 | —S(O)— | 0 | —(CH₂)₂—CH═CF₂ |
| 11 | —S(O)₂— | 2 | —(CH₂)₂—CF═CF₂ |
| 13 | —S(O)₂— | 0 | —(CH₂)₂—CF═CF₂ |
| 14 | —S(O)₂— | 0 | —(CH₂)₂—CH═CF₂ |
| 21 | —C(O)O— | 1 | —(CH₂)₂—CF═CF₂ |
| 22 | —C(O)O— | 1 | —(CH₂)₂—CH═CF₂ |
| 29 | —S(O)— | 1 | —(CH₂)₂—CF═CF₂ |
| 30 | —S(O)— | 1 | —(CH₂)₂—CH═CF₂ |
| 33 | —S(O)₂— | 1 | —(CH₂)₂—CF═CF₂ |
| 34 | —S(O)₂— | 1 | —(CH₂)₂—CH═CF₂ |
| 60 | —C(O)—N(Me)—S(O)₂— | 1 | —(CH₂)₂—CF═CF₂ |
| 61 | —C(O)—N(Me)—S(O)₂— | 1 | —(CH₂)₂—CH═CF₂ |
| 67 | —C(O)—NH—S(O)₂— | 1 | —(CH₂)₂—CF═CF₂ |
| 68 | —C(O)—NH—S(O)₂— | 1 | —(CH₂)₂—CH═CF₂ |
| 74 | —C(O)—NH—S(O)₂— | 2 | —(CH₂)₂—CF═CF₂ |
| 75 | —C(O)—NH—S(O)₂— | 2 | —(CH₂)₂—CH═CF₂ |
| 76 | —C(S)—NH—S(O)₂— | 1 | —(CH₂)₂—CF═CF₂ |
| 77 | —C(S)—NH—S(O)₂— | 1 | —(CH₂)₂—CH═CF₂ |

The compounds having general formula (I) can be prepared according to the processes illustrated herein.

A=—S(O)—, —S(O)₂—

In particular, the compounds having general formula (I), with A representing an —S(O)— group (compound having general formula (Ia)) or an —S(O)₂— (compound having general formula (Ib)) can be prepared from the respective thioethers having general formula (Ic) by oxidation reactions as indicated in scheme 1.

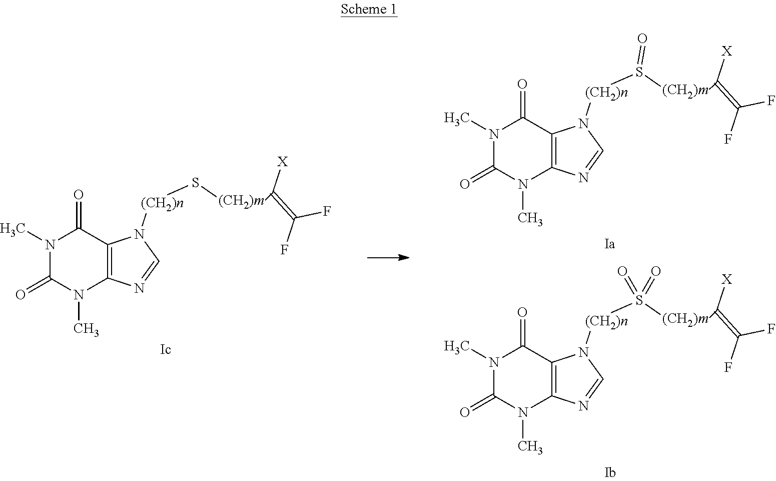

The reaction conditions, as indicated in WO 02/062770 and WO 2017/002100, provide for the use of an oxidizing agent in a suitable solvent. Oxidizing agents that can be used are perbenzoic acids such as 4-chloroperbenzoic acid, peracetic acid or inorganic peroxides, such as, for example, hydrogen peroxide, hydrogen peroxide-urea adduct and other oxidizing agents such as potassium permanganate, sodium periodate or potassium peroxymonosulfate. The solvents used are preferably halogenated hydrocarbons, such as dichloromethane, chloroform or dichloroethane, ethers such as dioxane or tetrahydrofuran, amides such as N,N-dimethylformamide or N-methylpyrrolidone, alcohols such as methanol, ethanol, propanol, isopropanol, ketones such as, for example, acetone, 2-butanone, organic acids such as acetic acid and water or mixtures thereof.

The reaction is carried out at a temperature ranging from 0° C. to the boiling point of the solvent, for a time ranging from 1 to 72 hours.

A=—S—

According to a first embodiment, the compounds having general formula (I), wherein A represents a sulfur atom (compound having formula (Ic)), can be prepared according to scheme 2, by reaction of the thiol derivative having formula (II) and compounds having formula (III), as indicated in WO 03/037878 and WO 2017/002100.

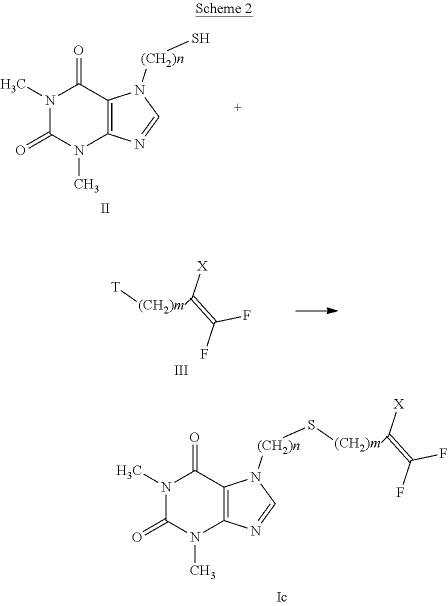

The reaction involves a substitution between the compound having formula (II) and the compound having formula (III), wherein T represents a leaving group, such as, for example, a halogen atom selected from Cl, Br or I, or a p-toluene sulfonate group, trifluoromethanesulfonate or methanesulfonate, and X is as defined in formula (I), in the presence of an organic or inorganic base such as, for example, triethylamine, pyridine, diisopropylethylamine, dim ethyl aminopyridine, sodium acetate, potassium or sodium bicarbonate, potassium or sodium carbonate, sodium or potassium or lithium hydroxide, in a suitable solvent such as, for example, halogenated hydrocarbons such as dichloromethane, dichloroethane, chloroform or amides such as N,N-dimethylformamide or N-methylpyrrolidone, ethers such as dioxane or tetrahydrofuran, nitriles such as acetonitrile, ketones such as acetone or 2-butanone, alcohols such as ethanol, methanol or isopropanol, or a mixture of halogenated or non-halogenated solvent and water.

This reaction is carried out at a temperature ranging from 0° C. to the boiling point of the solvent or mixture of solvents, for a time ranging from 1 to 96 hours. The compounds having general formula (II) can be obtained according to the syntheses indicated in scheme 3.

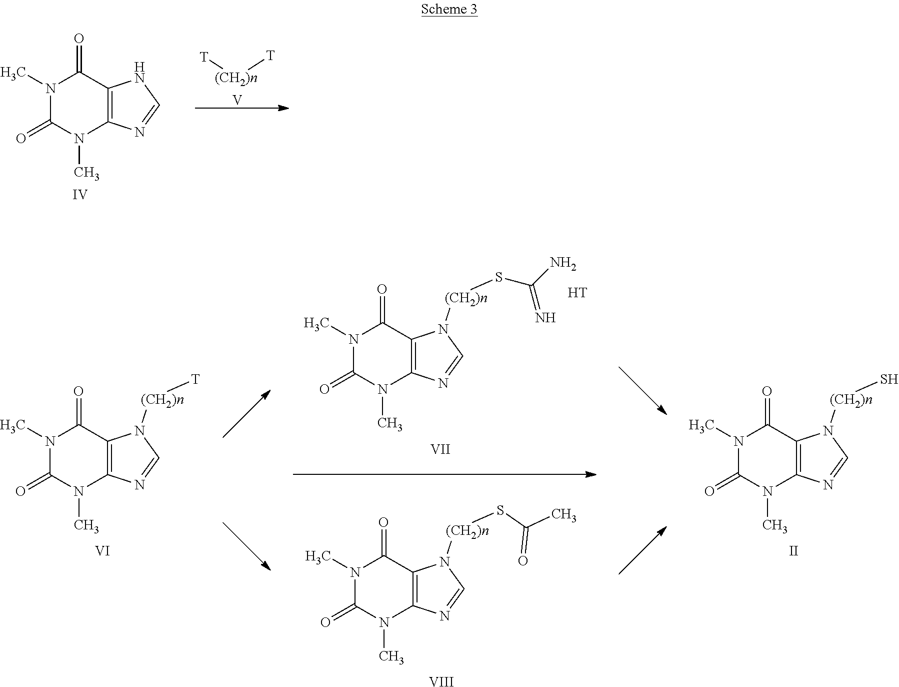

Compound (IV), called theophylline, is commercially available, and can be transformed into the intermediate (VI) by reaction with compounds having formula (V); these compounds having formula (V), which carry a leaving group T such as a halogen atom, selected from Cl, Br or I, or a p-toluenesulfonate, trifluoromethanesulfonate or methanesulfonate group, can be both commercially available and obtainable from the respective dialcohols by means of methods well-known in organic chemistry, as indicated in Theodora W. Greene, “PROTECTIVE GROUPS in ORGANIC SYNTHESIS”, Third Edition.

The reaction between the theophylline having formula (IV) and the compounds having formula (V) to give the intermediates having formula (VI) can be carried out in the presence of an organic or inorganic base such as, for example, triethylamine, pyridine, diisopropylethylamine, dimethylaminopyridine, sodium acetate, potassium or sodium bicarbonate, potassium or sodium carbonate, sodium or potassium or lithium hydroxide, alkaline metal hydrides such as sodium, lithium or potassium hydride, in a suitable solvent such as, for example, halogenated hydrocarbons such as dichloromethane, dichloroethane, chloroform or amides such as N,N-dimethylformamide or N-methylpyrrolidone, ethers such as dioxane or tetrahydrofuran, nitriles such as acetonitrile, ketones such as, for example, acetone or 2-butanone.

The compound having general formula (VI) can in turn be transformed directly into the intermediate thiouronium salt (VII) by reaction with thiourea in solvents such as alcohols, such as, for example, ethanol, methanol, isopropanol, propanol, or water or mixtures thereof, at a temperature ranging from 0° C. to the boiling point of the solvent. The subsequent hydrolysis in situ is carried out in solvents such as alcohols, water or mixtures thereof, tetrahydrofuran, dioxane, toluene, in the presence or absence of inorganic bases such as sodium or potassium carbonate and bicarbonate, sodium or potassium or lithium hydroxide, at room temperature.

According to a further synthesis method, the compounds having formula (II) can be prepared by reaction between the compounds having formula (VI) and the sodium or potassium salts of thioacetic acid, to obtain the respective thioesters having formula (VIII), in solvents such as N,N-dimethylformamide or N-methylpyrrolidone, dioxane or tetrahydrofuran, acetonitrile, at a temperature ranging from 0° C. to the respective boiling points. The subsequent hydrolysis is carried out in solvents such as alcohols, water or mixtures thereof, tetrahydrofuran, dioxane, toluene, in the presence or absence of inorganic bases such as sodium or potassium carbonate and bicarbonate, sodium or potassium or lithium hydroxide, at room temperature.

The above compounds having formula (II) can also be obtained starting from the corresponding thioesters having formula (VIII) by transacetylation reaction with reagents such as sodium or potassium methylate or ethylate, in solvents such as methanol or ethanol, at room temperature.

According to a further embodiment, the compounds having formula (Ic) can be prepared by treating compounds having formula (VI) with compounds having formula (IX), as indicated in scheme 4.

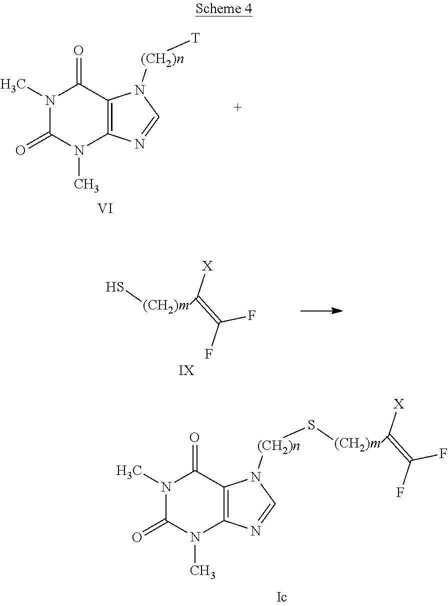

The reaction provides for the use of a compound having formula (VI), wherein T has the meanings described above and a compound having formula (IX) wherein X is as defined in formula (I), in the presence of an organic or inorganic base such as, for example, triethylamine, pyridine, diisopropylethylamine, dimethylaminopyridine, sodium acetate, potassium or sodium bicarbonate, potassium or sodium carbonate, sodium or potassium or lithium hydroxide, alkaline metal hydrides such as sodium, potassium or lithium hydride, in a suitable solvent such as, for example, halogenated hydrocarbons such as dichloromethane, dichloroethane, chloroform or amides such as N, N-dimethylformamide or N-methylpyrrolidone, ethers such as dioxane or tetrahydrofuran, nitriles such as acetonitrile, ketones such as acetone or 2-butanone, at a temperature ranging from 0° C. to the boiling point of the solvent, for a time ranging from 1 to 96 hours.

The compounds having formula (IX) can be easily obtained from the corresponding compounds having formula (III) using the same methods described above for the transformation of the compounds having formula (VI) into the compounds having general formula (II).

A=—C(O)O—

According to an embodiment, the compounds having general formula (I), wherein A represents a —C(O)O— group (compound having general formula (Id)), can be prepared from the corresponding acid having general formula (X) by esterification reaction with a suitable alcohol having general formula (XI) wherein X is as defined in formula (I), as indicated in reaction scheme 5, according to methods well-known in organic chemistry.

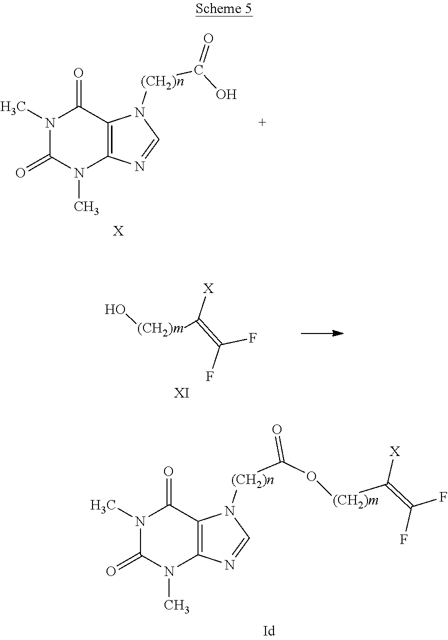

The reaction conditions provide for the use of a condenser such as N,N-dicyclohexylcarbodiimide, in the presence or absence of an amine such as N,N-dimethylaminopyridine, in an appropriate solvent such as dichloromethane, chloroform, tetrahydrofuran or dioxane at a temperature ranging from 0° C. to the boiling point of the solvent for a time ranging from 1 to 72 hours.

Alternatively, the compounds having formula (Id) can be obtained by reaction of the acid having formula (X) with the alcohol having formula (XI) in the presence of an acid catalysis using for example hydrochloric acid or sulfuric acid as described in R. C. Larock “Comprehensive Organic Transformations” or for example in F. T. Schevenels, M. Shen. A. Scott “J. American Chemical Society”, 2017, vol. 139 pages 6329-6337.

According to a further embodiment, the above-mentioned compounds having formula (Id) can be obtained by Mitsunobu reaction between the acid having formula (X) and the alcohol having general formula (XI) in the presence of triphenylphosphine and diethylazodicarboxylate in a solvent such as for example tetrahydrofuran, diethyl ether or dioxane at a temperature ranging from room temperature to the reflux temperature of the solvent, as described for example in U.S. Pat. No. 7,601,849.

According to another embodiment, the compounds having formula (Id) can be obtained by activating the carboxylic acid having formula (X) or via acyl chloride or via mixed anhydride to give the compound having formula (XII) wherein V represents a chlorine atom or an OCORW residue, with RW having the meaning of linear or branched C₁-C₄ alkyl, and the subsequent addition of the appropriate alcohol having general formula (XI) wherein X is as defined in formula (I), according to reaction scheme 6.

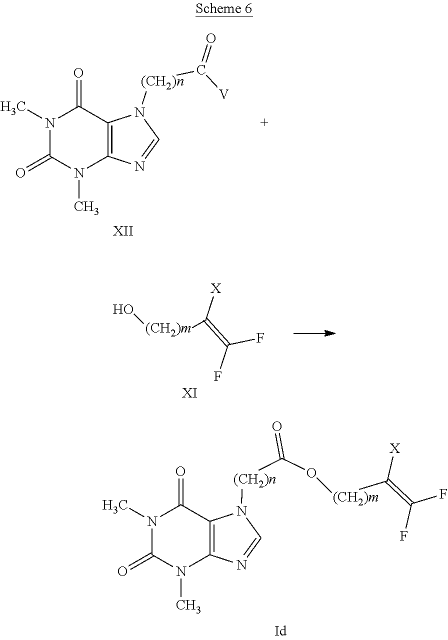

The reaction is carried out by reacting a compound having formula (XII), obtained from the compound having general formula (X) using methods well-known in literature, with an alcohol having general formula (XI) in the presence of a base such as triethylamine, N-methyl-morpholine or pyridine in a suitable solvent such as methylene chloride, chloroform or tetrahydrofuran at a temperature ranging from 0° C. to the boiling point of the solvent for a time ranging from 1 to 72 hours as widely described in R. C. Larock “Comprehensive Organic Transformations” or for example in Boutevin, A., Petrova K., Journal of Fluorine Chemistry, 1998, vol. 92, #1, pages 69-76 or in US 2004/198702.

The alcohols having general formula (XI) represent commercially available products or alternatively they can be synthesized according to procedures indicated in Fort et al., Angew. Chem. Int. Ed., 51: 10169-10172, Wang et al., Journal of Fluorine Chemistry 129 (2008) 607-612 or in US2012289738. According to a further embodiment, the compounds having general formula (Id) can be obtained from a suitable salt having formula (XIII) of the carboxylic acid having general formula (X), wherein M represents an alkaline metal, such as sodium, lithium or potassium, or an ammonium group, such as trimethylammonium or triethylammonium, in the presence of a derivative having formula (III) wherein T represents a leaving group such as a halogen atom, selected from chlorine, bromine or iodine or a trifluoromethanesulfonate or p-toluene sulfonate, or methanesulfonate group and X is as defined in formula (I), according to reaction scheme 7.

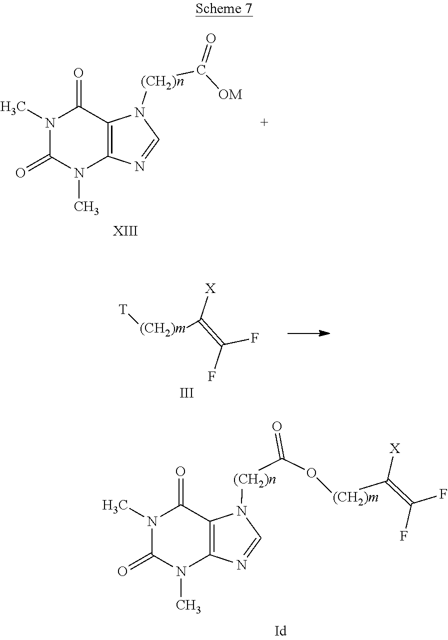

The reaction provides for the salification of the carboxylic acid having formula (X) with a base such as sodium bicarbonate, potassium bicarbonate, potassium carbonate, sodium carbonate, sodium hydroxide, potassium hydroxide, lithium hydroxide, sodium hydride or potassium t-butylate, triethylamine, diisopropylamine, in a solvent such as tetrahydrofuran, N,N-dimethylformamide, N-methylpyrrolidone, toluene or acetone, acetonitrile and the subsequent addition of a compound having formula (III) at a temperature ranging from room temperature to the reflux temperature of the solvent selected, as described for example in U.S. Pat. No. 5,519,015.

The compounds having formula (III) represent commercially available products. The compounds having formula (XIII) can be synthesized by hydrolysis of the respective esters having formula (XIV) with Rw having the meanings indicated above, in solvents such as water or tetrahydrofuran, dioxane or mixtures thereof, in the presence or absence of inorganic bases such as sodium or potassium carbonate and bicarbonate, sodium, potassium or lithium hydroxide, at room temperature, or in the presence of inorganic acids such as hydrochloric acid, sulfuric acid, hydrobromic acid, hydrogen iodide, or organic acids such as formic acid, acetic acid, trifluoroacetic acid, p-toluenesulfonic acid, according to what is indicated in Theodora W. Greene, “PROTECTIVE GROUPS in ORGANIC SYNTHESIS”, Third Edition (scheme 8).

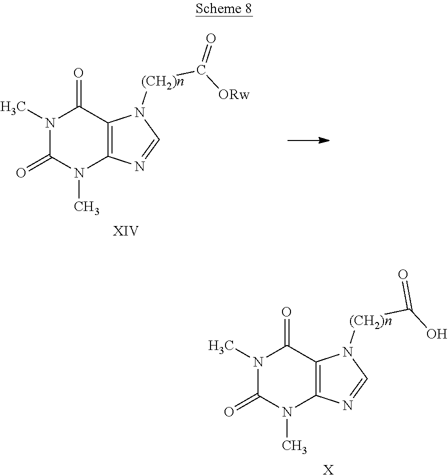

The esters having formula (XIV) can be prepared according to procedures indicated in Ruddarraju et al., European Journal of Medicinal Chemistry, 2016, vol. 123, pages 379-396, or in WO2013/23102.

A=—C(S)O—

According to an embodiment, the compounds having general formula (I), wherein A represents a —C(S)O— group (compound having general formula (Ie), with Y═S), can be prepared from the corresponding ester having general formula (Id) by reaction with phosphorus pentasulfide or with the Lawesson reagent, as indicated in scheme 9, according to procedures well-known in organic synthesis.

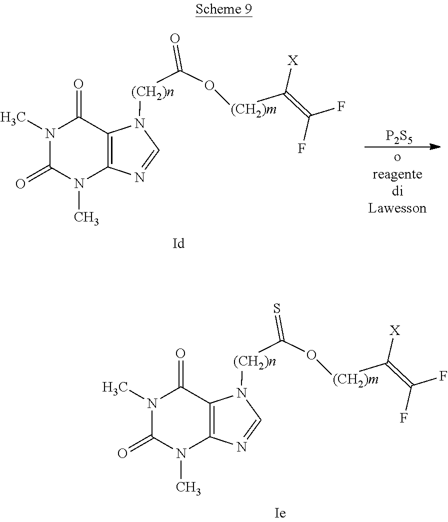

A=—C(O)—NR₁—S(O)₂—

The compounds having general formula (I), with A representing the group —C(Y)—NR₁—S(O)₂—, wherein Y represents an oxygen atom and R₁ a hydrogen atom, a C₁-C₆ alkyl group, a C₁-C₆ haloalkyl group, a C₂-C₆ alkenyl group, a C₂-C₆ haloalkenyl group, a C₃-C₈ cycloalkyl group, a C₄-C₉ cycloalkylalkyl group (compounds having general formula (If)), can be prepared by reaction of the respective carboxylic acids having formula (X) with sulfonamides having formula (XV) wherein E is as defined in formula (I), as illustrated in scheme 10.

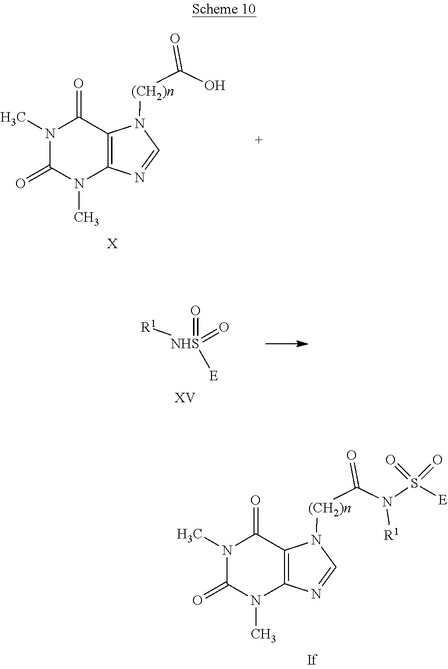

The reaction conditions provide for the use of a condensing agent, in the presence or absence of an amine such as N,N-dimethylaminopyridine in solvents such as alcohols, ethers, esters, amides or halogenated hydrocarbons or mixtures thereof. Condensing agents are for example 1-ethyl-3-(3-dimethylaminopropyl)-carbodiimide or its salts, N,N-dicyclohexylcarbodiimide, N,N-diisopropylcarbodiimide or 1,1′-carbonyldiimidazole.

The reaction is carried out at a temperature ranging from 0° C. to the boiling point of the solvent for a time ranging from 1 to 72 hours.

The compounds having general formula (If), with R₁ representing a C₁-C₆ alkyl group, a C₁-C₆ haloalkyl group, a C₂-C₆ alkenyl group, a C₂-C₆ haloalkenyl group, a C₃-C₈ cycloalkyl group, a C₄-C₉ cycloalkylalkyl group, can also be prepared by reaction between the compounds having general formula (If) (when R₁ represents a hydrogen atom) and appropriate halides, in the presence or absence of an organic or inorganic base, such as for example potassium or sodium carbonate, potassium or sodium bicarbonate, potassium or sodium hydroxide, potassium or sodium hydride, triethylamine, in solvents such as ethers or amides, such as for example tetrahydrofuran, dioxane or N,N-dimethylformamide. The halides used can be chlorides, iodides or bromides, commercial or obtainable from the respective alcohols using well-known organic chemistry techniques.

Alternatively, said compounds having general formula (If) can be prepared by reaction between compounds having formula (XII) and sulfonamides having formula (XV), as illustrated in scheme 11.

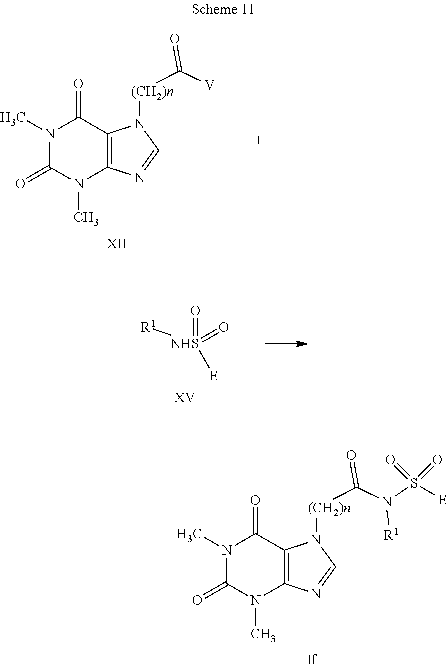

The compounds having formula (XII), wherein V represents a chlorine atom or a —OC(O)Rw residue, with Rw having the meaning of C₁-C₄ alkyl, are obtained from compounds having general formula (X) using methods known in literature; these carboxylic acid derivatives are then converted into the compounds having formula (If) by reaction with the sulfonamides having formula (XV) wherein E is as defined in formula (I), in the presence of a base such as triethylamine, N-methyl-morpholine or pyridine in a suitable solvent such as methylene chloride, chloroform or tetrahydrofuran at a temperature ranging from 0° C. to the boiling point of the solvent for a time ranging from 1 to 72 hours as widely described in RC Larock “Comprehensive Organic Transformations” or for example in Pan et al., European Journal of Medicinal Chemistry, 2012, vol. 50, pages 18 or in WO2010/129500.

The sulfonamides having general formula (XV) are structures known in literature and can be commercially available or can be prepared according to methods well-known in organic chemistry or according to what is indicated in WO2010/129500. The compounds having formula (If), when R₁ represents a —C(Y)R₂ group, a —C(O)OR₂ group, a —C(O)NR₂R₃ group, an —S(O)rR₂ group, an —S(O)₂NR₂R₃ group, can be synthesized by reaction of the compounds having general formula (If) (when R₁ represents a hydrogen atom), with acyl, sulfonyl or sulfinyl chlorides, for example ClC(Y)R₂, ClC(O)OR₂, ClC(O)NR₂R₃, ClS(O)rR₂, ClS(O)₂NR₂R₃, by means of acylation, sulfonylation or sulfinylation methods well-known in literature.

A=—C(S)—NR₁—S(O)₂—

The compounds having general formula (I), with A representing the group —C(Y)—NR₁—S(O)₂—, wherein Y represents a sulfur atom and R₁ represents a hydrogen atom, a C₁-C₆ alkyl group, a C₁-C₆ haloalkyl group, a C₂-C₆ alkenyl group, a C₂-C₆ haloalkenyl group, a C₃-C₈ cycloalkyl group or a C₄-C₉ cycloalkylalkyl group, (compounds having general formula (Ig)), can be prepared starting from the compounds having general formula (If) by reaction with phosphorus pentasulfide or with the Lawesson reagent, as shown in scheme 12, according to procedures well-known in organic synthesis.

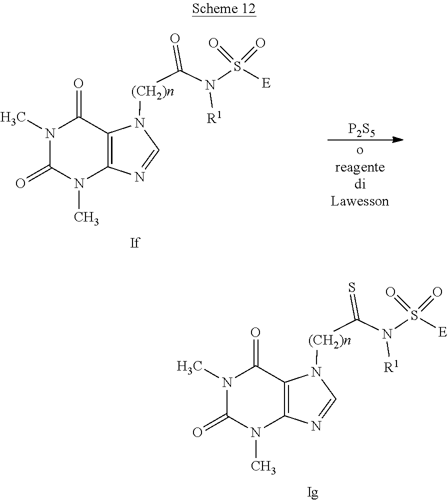

The compounds having formula (I), with A representing the group —C(Y)—NR₁—S(O)₂—, wherein Y represents a sulfur atom and R₁ represents a —C(Y)R₂ group, a —C(O)OR₂ group, a —C(O)NR₂R₃ group, an —S(O)rR₂ group, an —S(O)₂NR₂R₃ group, can be synthesized by reaction of the compounds having general formula (Ig), with R₁ representing a hydrogen atom, with acyl, sulfonyl or sulfinyl chlorides, for example ClC(Y)R₂, ClC(O)OR₂, ClC(O)NR₂R₃, ClS(O)rR₂, ClS(O)₂NR₂R₃, by means of acylation, sulfonylation or sulfinylation methods well-known in literature.

The compounds having general formula (I), for particular meanings of A, can be obtained in racemic form or as optically active isomers.

Considering both the compounds having general formula (I) isomerically pure, and mixtures thereof, possibly obtained during the preparation of the compounds having general formula (I) or deriving from an incomplete separation of the isomers themselves, in any proportion, therefore falls within the spirit of the present invention.

As already mentioned, the compounds having general formula (I) are provided with a high nematocidal activity and at effective doses, they do not exhibit phytotoxicity towards the application crops making them suitable for use in agriculture in the defence against nematodes.

In particular, the compounds object of the present invention are effective in the control of numerous nematodes such as, for example: Pratylenchus spp, Globodera spp, Heterodera spp, Meloidogyne spp, Aphelenchoides spp, Radopholus similis, Ditylenchus dipsaci, Tylenchulus semipenetrans, Longidorus spp, Xiphinema spp, Trichodorus spp, Bursaphelenchus spp, Belonolaimus spp, and the like.

A second object of the present invention relates to a method for the control of nematodes in cultivated areas by applying at least one compound having general formula (XVI) to the plant and/or to the soil,

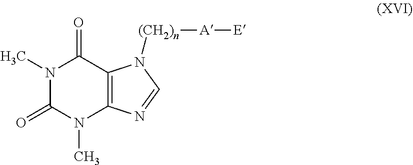

wherein:
n represents an integer ranging from 0 to 3;
A′ represents a group selected from: —S—, —S(O)—, —S(O)₂—, —C(═Y)—O—, —C(═Y)—NR₁—, —C(═Y)—NR₁—S(O)₂—;
Y represents an oxygen or sulfur atom;
R₁ represents a hydrogen atom, a C₁-C₆ alkyl group, a C₁-C₆ haloalkyl group, a C₂-C₆ alkenyl group, a C₂-C₆ haloalkenyl group, a C₃-C₈ cycloalkyl group, a C₄-C₉ cycloalkylalkyl group, a C(═Y)R₂ group, a C(═O)OR₂ group, a C(═O)NR₂R₃ group, an S(═O)ᵣR₂ group, an S(═O)₂NR₂R₃ group;
R₂ and R₃, the same as or different from each other, represent a hydrogen atom, a C₁-C₆ alkyl group, a C₁-C₆ haloalkyl group, a C₂-C₆ alkenyl group, a C₂-C₆ haloalkenyl group, a C₃-C₈ cycloalkyl group, a C₃-C₈ halocycloalkyl group, an aryl group, a benzyl group;
r represents an integer ranging from 0 to 2;
E′ represents a group —(CH₂)ₘ—CX═CF₂, or an optionally substituted group selected from a C₁-C₆ alkyl, a C₁-C₆ haloalkyl group, a C₂-C₆ alkenyl group, a C₂-C₆ haloalkenyl group, a C₂-C₆ alkynyl group, a C₂-C₆ haloalkynyl group, a C₃-C₈ cycloalkyl group, a C₃-C₈ halocycloalkyl group, a C₄-C₉ cycloalkylalkyl group, an aryl group, a naphthyl group, a C₇-C₁₄ arylalkylene group, a heterocyclic group, penta- or hexa-atomic, aromatic or non-aromatic, also benzocondensed or heterobicyclic, containing at least one heteroatom selected from oxygen, sulfur, nitrogen, optionally oxidized to N-oxide, or a C₃-C₉ heterocyclylalkylene group wherein the heterocyclic group is as defined above;
m represents an integer ranging from 1 to 6;
X represents a hydrogen or fluorine atom;

with the proviso that when A′ represents a group selected from: —S—, —S(═O)—, —S(═O)₂—, —C(═Y)—O—, then E′ represents the group —(CH₂)ₘ—CX═CF₂. Said C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₃-C₈ cycloalkyl, C₃-C₈ halocycloalkyl, C₄-C₉ cycloalkylalkyl, aryl, naphthyl, benzyl, arylalkylene, heterocyclic or heterocyclylalkylene groups can be non-substituted or substituted by one or more Q group(s) selected from halogen, C C₃-C₆-cycloalkyl, C₄-C₉-cycloalkylalkyl, C₃-C₆-halocycloalkyl, C₁-C₆-haloalkoxyl, C₁-C₆-thiohaloalkoxyl, C₁-C₆-alkylsulfinyl, C₁-C₆-alkyl sulfonyl, C₁-C₆-alkoxycarbonyl, C₃-C₆-cycloalkoxycarbonyl, amino, N—C₁-C₆-alkylamino, N,N—C₂-C₁₂-dialkylamino, N—C₁-C₆-alkoxycarbo-nylamino, N—C₃-C₆-cycloalkyl amino, N,N—C₆-C₁₂-dicyclo-alkylamino, N—C₃-C₆-cycloalkoxycarbonylamino, C₁-C₆-alkylaminocarbonyl, C₃-C₆-cycloalkylaminocarbonyl, a NR₂R₃CONR₂— group; formyl, C₁-C₆-alkylcarbonyl, carboxyl, cyano, an optionally substituted aryl, a benzyl, a heterocycle, optionally substituted, penta- or hexa-atomic, aromatic or non-aromatic, also benzocondensed or heterobicyclic, containing at least one heteroatom selected from oxygen, sulfur, nitrogen, optionally oxidized to N-oxide, a (C₁-C₆)-alkyl-heterocyclic group, optionally substituted, penta- or hexa-atomic, also benzocondensed or heterobicyclic, containing at least one heteroatom selected from oxygen, sulfur, and nitrogen, optionally oxidized to N-oxide.

The compounds having general formula (XVI), wherein A′ represents the group —C(═Y)—NR₁, are represented by the following formula (Ih):

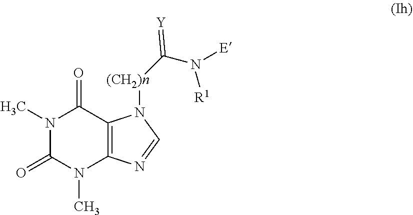

The compounds having general formula (XVI) can be prepared according to the processes illustrated herein.

The compounds having formula (XVI), wherein A′ represents the group —C(═Y)—NR₁— and Y has the meaning of oxygen, can be easily obtained from the corresponding compounds having general formula (XII) or formula (X) according to methods well-known in literature and described for example in RC Larock “Comprehensive Organic Transformations” or in WO2015028427 or WO2018108791.

The compounds having formula (XVI), wherein A′ represents the group —C(═Y)—NR₁— and Y has the meaning of sulfur, can be prepared according to what described above for the transformation of compounds (If) into the compounds having general formula (Ig) (Scheme 12).

The compounds having general formula (XVI), with A′ representing a —C(Y)—NR₁—S(O)₂— group, wherein Y represents an oxygen atom, can be prepared according to what is described above for the preparation of compounds having general formula (If) (Schemes 10 and 11).

The compounds having general formula (XVI), with A′ representing a —C(Y)—NR₁—S(O)₂ group, wherein Y represents a sulfur atom, can be prepared according to what is described above for the transformation of compounds (If) into compounds having general formula (Ig) (Scheme 12).

The compounds having general formula (XVI), with A′ representing a —C(═Y)—O— group, wherein Y represents an oxygen atom, can be prepared according to what is described with reference to the compounds having general formula (Id) (Scheme 5, 6 or 7).

The compounds having general formula (XVI), with A′ representing a —C(═Y)—O— group, wherein Y represents a sulfur atom, can be prepared according to what is described with reference to the compounds having general formula (Ie), (Scheme 9).

The compounds having general formula (XVI), with A′ representing an —S(═O)—, —S(═O)₂— group can be prepared according to what is described with reference to the compounds having general formula (Ia) and (Ib) respectively (Scheme 1). The compounds having general formula (XVI), with A′ representing an —S— group, can be prepared according to what is described with reference to the compounds having general formula (Ic) (Scheme 2 or 4).

In particular, this method allows the prevention and treatment of phytoparasitosis caused by nematodes.

The compounds having general formula (XVI) in the present method can be effective in several stages of the development of the parasite, for example in reducing the hatching of the eggs, in killing the larvae, in inhibiting the population growth rate, in killing the adults.

The compounds having general formula (XVI) can be applied at different times of the vegetative development of the plant, for example before transplanting/sowing or during the growth of the plant, via the leaves, or to the soil by fertigation, or incorporation in the ground, or through seed tanning. The quantity of compound to be applied for obtaining the desired effect can vary according to various factors such as, for example, the compound used, the crop to be protected, the degree of infestation, the climatic conditions, the characteristics of the soil, the application method, etc.

Doses of compound ranging from 100 g to 10,000 g per hectare generally provide sufficient control.

Preferred doses range from 500 g to 5,000 g per hectare.

Specific examples of compounds having general formula (XVI), useful for the prevention and treatment of phytoparasitosis caused by nematodes, are compounds wherein n, A ‘and E’ have the meanings indicated in table 3:

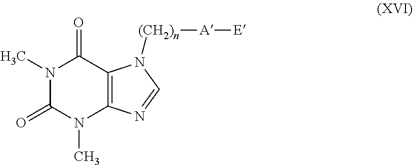

TABLE 3

| Comp. | | | |
|---|---|---|---|
| Nr | A′ | n | E′ |
| 1 | —C(O)O— | 0 | —(CH₂)₂—CF═CF₂ |
| 2 | —C(O)O— | 0 | —(CH₂)₂—CH═CF₂ |
| 3 | —C(O)O— | 0 | —(CH₂)₄—CF═CF₂ |
| 4 | —C(O)O— | 2 | —(CH₂)₂—CF═CF₂ |
| 5 | —S— | 0 | —(CH₂)₂—CF═CF₂ |
| 6 | —S— | 0 | —(CH₂)₂—CH═CF₂ |
| 7 | —S— | 2 | —(CH₂)₂—CF═CF₂ |
| 8 | —S(O)— | 2 | —(CH₂)₂—CF═CF₂ |
| 9 | —S(O)— | 0 | —(CH₂)₂—CF═CF₂ |
| 10 | —S(O)— | 0 | —(CH₂)₂—CH═CF₂ |
| 11 | —S(O)₂— | 2 | —(CH₂)₂—CF═CF₂ |
| 12 | —S(O)— | 0 | —(CH₂)₄—CH═CF₂ |
| 13 | —S(O)₂— | 0 | —(CH₂)₂—CF═CF₂ |
| 14 | —S(O)₂— | 0 | —(CH₂)₂—CH═CF₂ |
| 15 | —S(O)₂— | 0 | —(CH₂)₄—CF═CF₂ |
| 16 | —S(O)₂— | 0 | —(CH₂)₄—CH═CF₂ |
| 17 | —C(S)O— | 0 | —(CH₂)₂—CF═CF₂ |
| 18 | —C(S)O— | 0 | —(CH₂)₂—CH═CF₂ |
| 19 | —C(S)O— | 0 | —(CH₂)₄—CF═CF₂ |
| 20 | —C(S)O— | 0 | —(CH₂)₄—CH═CF₂ |
| 21 | —C(O)O— | 1 | —(CH₂)₂—CF═CF₂ |
| 22 | —C(O)O— | 1 | —(CH₂)₂—CH═CF₂ |
| 23 | —C(O)O— | 1 | —(CH₂)₄—CF═CF₂ |
| 24 | —C(O)O— | 1 | —(CH₂)₄—CH═CF₂ |
| 25 | —S— | 1 | —(CH₂)₂—CF═CF₂ |
| 26 | —S— | 1 | —(CH₂)₂—CH═CF₂ |
| 27 | —S— | 1 | —(CH₂)₄—CF═CF₂ |
| 28 | —S— | 1 | —(CH₂)₄—CH═CF₂ |
| 29 | —S(O)— | 1 | —(CH₂)₂—CF═CF₂ |
| 30 | —S(O)— | 1 | —(CH₂)₂—CH═CF₂ |
| 31 | —S(O)— | 1 | —(CH₂)₄—CF═CF₂ |
| 32 | —S(O)— | 1 | —(CH₂)₄—CH═CF₂ |
| 33 | —S(O)₂— | 1 | —(CH₂)₂—CF═CF₂ |
| 34 | —S(O)₂— | 1 | —(CH₂)₂—CH═CF₂ |
| 35 | —S(O)₂— | 1 | —(CH₂)₄—CF═CF₂ |
| 36 | —S(O)₂— | 1 | —(CH₂)₄—CH═CF₂ |
| 37 | —C(S)O— | 1 | —(CH₂)₂—CF═CF₂ |
| 38 | —C(S)O— | 1 | —(CH₂)₂—CH═CF₂ |
| 39 | —C(S)O— | 1 | —(CH₂)₄—CF═CF₂ |
| 40 | —C(S)O— | 1 | —(CH₂)₄—CH═CF₂ |
| 41 | —C(O)—NH—S(O)₂— | 0 | —(CH₂)₂—CF═CF₂ |
| 42 | —C(O)—NH—S(O)₂— | 0 | —(CH₂)₂—CH═CF₂ |
| 43 | —C(O)—NH—S(O)₂— | 0 | 2-propyl |
| 44 | —C(O)—NH—S(O)₂— | 0 | 2-methyl-1-propyl |
| 45 | —C(O)—NH—S(O)₂— | 0 | 2-methoxy-1-ethyl |
| 46 | —C(O)—NH—S(O)₂— | 0 | 2-trifluoro-1-ethyl |
| 47 | —C(O)—NH—S(O)₂— | 0 | 3-bromo-1-butyl |
| 48 | —C(O)—NH—S(O)₂— | 1 | 2-propyl |
| 49 | —C(O)—NH—S(O)₂— | 1 | 2-methyl-1-propyl |
| 50 | —C(O)—NH—S(O)₂— | 1 | 2-methoxy-1-ethyl |
| 51 | —C(O)—NH—S(O)₂— | 1 | 2-trifluoro-1-ethyl |
| 52 | —C(O)—NH—S(O)₂— | 1 | 3-bromo-1-butyl |
| 53 | —C(O)—N(Me)—S(O)₂— | 0 | —(CH₂)₂—CF═CF₂ |
| 54 | —C(O)—N(Me)—S(O)₂— | 0 | —(CH₂)₂—CH═CF₂ |
| 55 | —C(O)—N(Me)—S(O)₂— | 0 | 2-propyl |
| 56 | —C(O)—N(Me)—S(O)₂— | 0 | 2-trifluoro-1-ethyl |
| 57 | —C(O)—N(Me)—S(O)₂— | 0 | 2-methyl-1-propyl |
| 58 | —C(O)—N(Me)—S(O)₂— | 0 | 2-methoxy-1-ethyl |
| 59 | —C(O)—N(Me)—S(O)₂— | 0 | 3-bromo-1-butyl |
| 60 | —C(O)—N(Me)—S(O)₂— | 1 | —(CH₂)₂—CF═CF₂ |
| 61 | —C(O)—N(Me)—S(O)₂— | 1 | —(CH₂)₂—CH═CF₂ |
| 62 | —C(O)—N(Me)—S(O)₂— | 1 | 2-propyl |
| 63 | —C(O)—N(Me)—S(O)₂— | 1 | 2-trifluoro-1-ethyl |
| 64 | —C(O)—N(Me)—S(O)₂— | 1 | 2-methyl-1-propyl |
| 65 | —C(O)—N(Me)—S(O)₂— | 1 | 2-methoxy-1-ethyl |
| 66 | —C(O)—N(Me)—S(O)₂— | 1 | 3-bromo-1-butyl |
| 67 | —C(O)—NH—S(O)₂— | 1 | —(CH₂)₂—CF═CF₂ |
| 68 | —C(O)—NH—S(O)₂— | 1 | —(CH₂)₂—CH═CF₂ |
| 69 | —C(O)—NH—S(O)₂— | 2 | 2-propyl |
| 70 | —C(O)—NH—S(O)₂— | 2 | 2-trifluoro-1-ethyl |
| 71 | —C(O)—NH—S(O)₂— | 2 | 2-methyl-1-propyl |
| 72 | —C(O)—NH—S(O)₂— | 2 | 2-methoxy-1-ethyl |
| 73 | —C(O)—NH—S(O)₂— | 2 | 3-bromo-1-butyl |
| 74 | —C(O)—NH—S(O)₂— | 2 | —(CH₂)₂—CF═CF₂ |
| 75 | —C(O)—NH—S(O)₂— | 2 | —(CH₂)₂—CH═CF₂ |
| 76 | —C(S)—NH—S(O)₂— | 1 | —(CH₂)₂—CF═CF₂ |
| 77 | —C(S)—NH—S(O)₂— | 1 | —(CH₂)₂—CH═CF₂ |
| 78 | —C(S)—NH—S(O)₂— | 1 | 2-propyl |
| 79 | —C(S)—NH—S(O)₂— | 1 | 2-trifluoro-1-ethyl |
| 80 | —C(S)—NH—S(O)₂— | 1 | 2-methyl-1-propyl |
| 81 | —C(S)—NH—S(O)₂— | 1 | 2-methoxy-1-ethyl |
| 82 | —C(S)—N(Me)—S(O)₂— | 1 | —(CH₂)₂—CF═CF₂ |
| 83 | —C(S)—N(Me)—S(O)₂— | 1 | —(CH₂)₂—CH═CF₂ |
| 84 | —C(S)—N(Me)—S(O)₂— | 1 | 2-propyl |
| 85 | —C(S)—N(Me)—S(O)₂— | 1 | 2-trifluoro-1-ethyl |
| 86 | —C(S)—N(Me)—S(O)₂— | 1 | 2-methyl-1-propyl |
| 87 | —C(S)—N(Me)—S(O)₂— | 1 | 2-methoxy-1-ethyl |
| 88 | —C(S)—NH—S(O)₂— | 2 | —(CH₂)₂—CF═CF₂ |
| 89 | —C(S)—NH—S(O)₂— | 2 | —(CH₂)₂—CH═CF₂ |
| 90 | —C(S)—NH—S(O)₂— | 2 | 2-propyl |
| 91 | —C(S)—NH—S(O)₂— | 2 | 2-trifluoro-1-ethyl |
| 92 | —C(S)—NH—S(O)₂— | 2 | 2-methyl-1-propyl |
| 93 | —C(S)—NH—S(O)₂— | 2 | 2-methoxy-1-ethyl |
| 94 | —C(S)—N(Me)—S(O)₂— | 2 | —(CH₂)₂—CF═CF₂ |
| 95 | —C(S)—N(Me)—S(O)₂— | 2 | —(CH₂)₂—CH═CF₂ |
| 96 | —C(S)—N(Me)—S(O)₂— | 2 | 2-propyl |
| 97 | —C(S)—N(Me)—S(O)₂— | 2 | 2-trifluoro-1-ethyl |
| 98 | —C(O)—NH—S(O)₂— | 1 | 2-chloro-5-methoxy-phenyl |
| 99 | —C(O)—NH—S(O)₂— | 1 | 2,3-dimethyl-5-(1- |
| | | | pyrazolylmethyl)-phenyl |
| 100 | —C(O)—NH—S(O)₂— | 1 | 2-(ethylamino)-phenyl |
| 101 | —C(O)—NH—S(O)₂— | 1 | 2-chloro-5-thiazolyl |
| 102 | —C(O)—NH—S(O)₂— | 1 | 3,5-dimethyl-4-isoxazolyl |
| 103 | —C(O)—NH—S(O)₂— | 1 | 2-benzothienyl |
| 104 | —C(O)—NH—S(O)₂— | 1 | 3-chloro-2-thienyl |
| 105 | —C(O)—NH—S(O)₂— | 1 | 4-ethyl-3-pyridyl |
| 106 | —C(O)—N(Me)—S(O)₂— | 1 | 2-chloro-5-methoxy-phenyl |
| 107 | —C(O)—N(Me)—S(O)₂— | 1 | 2,3-dimethyl-5-(1- |
| | | | pyrazolylmethyl)-phenyl |
| 108 | —C(O)—N(Me)—S(O)₂— | 1 | 2-(ethylamino)-phenyl |
| 109 | —C(O)—N(Me)—S(O)₂— | 1 | 2-chloro-5-thiazolyl |
| 110 | —C(O)—N(Me)—S(O)₂— | 1 | 3,5-dimethyl-4-isoxazolyl |
| 111 | —C(O)—N(Me)—S(O)₂— | 1 | 2-benzothienyl |
| 112 | —C(O)—N(Me)—S(O)₂— | 1 | 3-chloro-2-thienyl |
| 113 | —C(O)—N(Me)—S(O)₂— | 1 | 4-ethyl-3-pyridyl |
| 114 | —C(O)—NH—S(O)₂— | 0 | 2-chloro-5-methoxy-phenyl |
| 115 | —C(O)—NH—S(O)₂— | 0 | 2-chloro-6-fluoro-phenyl |
| 116 | —C(O)—NH—S(O)₂— | 0 | 2-chloro-phenyl |
| 117 | —C(S)—NH—S(O)₂— | 0 | 2-chloro-5-methoxy-phenyl |
| 118 | —C(S)—NH—S(O)₂— | 0 | 2-chloro-6-fluoro-phenyl |
| 119 | —C(S)—NH—S(O)₂— | 0 | 2-chloro-phenyl |
| 120 | —C(S)—N(Me)—S(O)₂— | 0 | 2-chloro-5-methoxy-phenyl |
| 121 | —C(S)—N(Me)—S(O)₂— | 0 | 2-chloro-6-fluoro-phenyl |
| 122 | —C(S)—N(Me)—S(O)₂— | 0 | 2-chloro-phenyl |
| 123 | —C(S)—N(Me)—S(O)₂— | 0 | 2,4,6-trifluoro-phenyl |
| 124 | —C(S)—NH—S(O)₂— | 1 | 2-chloro-5-methoxy-phenyl |
| 125 | —C(S)—NH—S(O)₂— | 1 | 2-chloro-6-fluoro-phenyl |
| 126 | —C(S)—NH—S(O)₂— | 1 | 2-chloro-phenyl |
| 127 | —C(S)—N(Me)—S(O)₂— | 1 | 2-chloro-5-methoxy-phenyl |
| 128 | —C(S)—N(Me)—S(O)₂— | 1 | 2-chloro-6-fluoro-phenyl |
| 129 | —C(S)—N(Me)—S(O)₂— | 1 | 2-chloro-phenyl |
| 130 | —C(S)—N(Me)—S(O)₂— | 1 | 2,4,6-trifluoro-phenyl |
| 131 | —C(O)—NH— | 1 | —(CH₂)₂—CF═CF₂ |
| 132 | —C(O)—NH— | 1 | —(CH₂)₂—CH═CF₂ |
| 133 | —C(O)—NH— | 1 | 2-[3-chloro-5-(trifluoromethyl)- |
| | | | 2-pyridyl]-1-ethyl |
| 134 | —C(O)—NH— | 1 | 3-[(2-trifluoromethyl)-phenyl]- |
| | | | 1,2,4-thiadiazol-5-yl |
| 135 | —C(O)—NH— | 1 | 3-[(2,3,5,6-tetrafluoro)-phenyl]- |
| | | | 1,2,4-thiadiazol-5-yl |
| 136 | —C(O)—NH— | 1 | 5-[(2-trifluoromethyl)-phenyl- |
| | | | 1,3,4-thiadiazol-2-yl |
| 137 | —C(O)—NH— | 1 | 5-[(2,3,5,6-tetrafluoro)-phenyl]- |
| | | | 1,3,4-thiadiazol-2-yl |
| 138 | —C(O)—NH— | 1 | 2-chloro-5-methoxy-phenyl |
| 139 | —C(O)—NH— | 1 | 2-chloro-6-fluoro-phenyl |
| 140 | —C(O)—NH— | 1 | 2-chloro-phenyl |
| 141 | —C(O)—NH— | 1 | 2,4,6-trifluoro-phenyl |
| 142 | —C(O)—NH— | 0 | 2-[3-chloro-5-(trifluoromethyl)- |
| | | | 2-pyridyl]-1-ethyl |
| 143 | —C(O)—NH— | 0 | 3-[(2-trifluoromethyl)-phenyl]- |
| | | | 1,2,4-thiadiazol-5-yl |
| 144 | —C(O)—NH— | 0 | 3-[(2,3,5,6-tetrafluoro)-phenyl]- |
| | | | 1,2,4-thiadiazol-5-yl |
| 145 | —C(O)—NH— | 0 | 5-[(2-trifluoromethyl)-phenyl]- |
| | | | 1,3,4-thiadiazol-2-yl |
| 146 | —C(O)—NH— | 0 | 5-[(2,3,5,6-tetrafluoro)-phenyl]- |
| | | | 1,3,4-thiadiazol-2-yl |
| 147 | —C(O)—NH— | 0 | 2-chloro-5-methoxy-phenyl |
| 148 | —C(O)—NH— | 1 | 1-methyl-5-pyrazolyl |
| 149 | —C(O)—NH— | 1 | 4-ethyl-3-pyridyl |
| 150 | —C(O)—NH— | 1 | 2-thienyl |
| 151 | —C(O)—N(Me)— | 0 | —(CH₂)₂—CF═CF₂ |
| 152 | —C(O)—N(Me)— | 0 | —(CH₂)₂—CH═CF₂ |
| 153 | —C(O)—N(Me)— | 0 | 2,4-difluoro-phenyl |
| 154 | —C(O)—N(Me)— | 0 | 3-chloro-2-pyridyl |
| 155 | —C(O)—N(Me)— | 0 | 2-(trifluoromethoxy)-phenyl |
| 156 | —C(O)—N(Me)— | 0 | 2-chloro-5-trifluoromethyl- |
| | | | phenyl |
| 157 | —C(O)—N(Me)— | 0 | 2-chloro-5-methoxy-phenyl |
| 158 | —C(S)—NH— | 0 | —(CH₂)₂—CF═CF₂ |
| 159 | —C(S)—NH— | 0 | —(CH₂)₂—CH═CF₂ |
| 160 | —C(S)—NH— | 0 | 2-[3-chloro-5-(trifluoromethyl)- |
| | | | 2-pyridyl]-1-ethyl |
| 161 | —C(S)—NH— | 0 | 3-[(2-trifluoromethyl)-phenyl]- |
| | | | 1,2,4-thiadiazol-5-yl |
| 162 | —C(S)—NH— | 0 | 3-[(2,3,5,6-tetrafluoro)-phenyl]- |
| | | | 1,2,4-thiadiazol-5-yl |
| 163 | —C(S)—NH— | 0 | 5-[(2-trifluoromethyl)-phenyl]- |
| | | | 1,3,4-thiadiazol-2-yl |
| 164 | —C(S)—NH— | 0 | 5-[(2,3,5,6-tetrafluoro)-phenyl]- |
| | | | 1,3,4-thiadiazol-2-yl |
| 165 | —C(S)—NH— | 0 | 2-chloro-5-methoxy-phenyl |
| 166 | —C(S)—NH— | 1 | —(CH₂)₂—CF═CF₂ |
| 167 | —C(S)—NH— | 1 | —(CH₂)₂—CH═CF₂ |
| 168 | —C(S)—NH— | 1 | 2-[3-chloro-5-(trifluoromethyl)- |
| | | | 2-pyridyl]-1-ethyl |
| 169 | —C(S)—NH— | 1 | 3-[(2-trifluoromethyl)-phenyl]- |
| | | | 1,2,4-thiadiazol-5-yl |
| 170 | —C(S)—NH— | 1 | 3-[(2,3,5,6-tetrafluoro)-phenyl]- |
| | | | 1,2,4-thiadiazol-5-yl |
| 171 | —C(S)—NH— | 1 | 5-[(2-trifluoromethyl)-phenyl]- |
| | | | 1,3,4-thiadiazol-2-yl |
| 172 | —C(S)—NH— | 1 | 5-[(2,3,5,6-tetrafluoro)-phenyl]- |
| | | | 1,3,4-thiadiazol-2-yl |
| 173 | —C(S)—NH— | 1 | 2-chloro-5-methoxy-phenyl |
| 174 | —C(S)—NH— | 1 | 2-chloro-6-fluoro-phenyl |
| 175 | —C(S)—NH— | 1 | 2-chloro-phenyl |
| 176 | —C(S)—NH— | 1 | 2,4,6-trifluoro-phenyl |
| 177 | —C(S)—NH— | 1 | 4-pyridyl |
| 178 | —C(S)—NH— | 1 | 4-ethyl-3-pyridyl |
| 179 | —C(S)—NH— | 1 | 2-thienyl |
| 180 | —C(S)—N(Me)— | 0 | —(CH₂)₂—CF═CF₂ |
| 181 | —C(S)—N(Me)— | 0 | —(CH₂)₂—CH═CF₂ |
| 182 | —C(S)—N(Me)— | 0 | 2-[3-chloro-5-(trifluoromethyl)- |
| | | | 2-pyridyl]-1-ethyl |
| 183 | —C(S)—N(Me)— | 0 | 3-[(2-trifluoromethyl)-phenyl]- |
| | | | 1,2,4-thiadiazol-5-yl |
| 184 | —C(S)—N(Me)— | 0 | 3-[(2,3,5,6-tetrafluoro)-phenyl]- |
| | | | 1,2,4-thiadiazol-5-yl |
| 185 | —C(S)—N(Me)— | 0 | 5-[(2-trifluoromethyl)-phenyl]- |
| | | | 1,3,4-thiadiazol-2-yl |
| 186 | —C(S)—N(Me)— | 0 | 5-[(2,3,5,6-tetrafluoro)-phenyl]- |
| | | | 1,3,4-thiadiazol-2-yl |
| 187 | —C(S)—N(Me)— | 0 | 2-chloro-5-methoxy-phenyl |
| 188 | —C(S)—N(Me)— | 0 | 2-chloro-6-fluoro-phenyl |
| 189 | —C(S)—N(Me)— | 0 | 2-chloro-5-thiazolyl |
| 190 | —C(S)—N(Me)— | 0 | 3,5-dimethyl-4-isoxazolyl |
| 191 | —C(S)—N(Me)— | 0 | 1-cyclopropyl |
| 192 | —C(S)—N(Me)— | 0 | 2-cyclopentyl-6-methyl-phenyl |
| 193 | —C(O)—NH | 0 | 2-bromo-phenyl |
| 194 | —C(O)—N(Me)— | 0 | 2-bromo-phenyl |
| 195 | —C(O)—NH | 1 | 2-bromo-phenyl |
| 196 | —C(O)—N(Me)— | 1 | 2-bromo-phenyl |
| 197 | —C(S)—NH— | 1 | 2-bromo-phenyl |
| 198 | —C(S)—N(Me)— | 1 | 2-bromo-phenyl |
| 199 | —C(S)—N(Me)— | 1 | —(CH₂)₂—CF═CF₂ |
| 200 | —C(S)—N(Me)— | 1 | —(CH₂)₂—CH═CF₂ |
| 201 | —C(S)—N(Me)— | 1 | 2-chloro-5-methoxy-phenyl |
| 202 | —C(S)—N(Me)— | 1 | 2-chloro-6-fluoro-phenyl |
| 203 | —C(S)—N(Me)— | 1 | 2-chloro-phenyl |
| 204 | —C(S)—N(Me)— | 1 | 2,4,6-trifluoro-phenyl |
| 205 | —C(S)—N(Me)— | 1 | 3-chloro-1-benzyl |
| 206 | —C(S)—N(Me)— | 1 | 2,5-dimethoxy-phenyl |
| 207 | —C(S)—N(Me)— | 1 | 2,4-difluoro-phenyl |
| 208 | —C(S)—N(Me)— | 1 | 2-i-propyl-phenyl |
| 209 | —C(S)—N(Me)— | 1 | 1-naphthyl |
| 210 | —C(S)—N(Me)— | 1 | 2-(methylsulfonyl)-phenyl |
| 211 | —C(S)—N(Me)— | 1 | 3-chloro-2-pyridyl |
| 212 | —C(S)—N(Me)— | 1 | 2-(trifluoromethoxy)-phenyl |
| 213 | —C(S)—N(Me)— | 1 | 2-chloro-5-trifluoromethyl- |
| | | | phenyl |
| 214 | —C(S)—N(Me)— | 1 | 1-quinolyl |
| 215 | —C(S)—N(Me)— | 1 | 1-methyl-5-pyrazolyl |
| 216 | —C(S)—N(Me)— | 1 | 3,5-dimethyl-phenyl |
| 217 | —C(S)—N(Me)— | 1 | 2-cyanophenyl |
| 218 | —C(S)—N(Me)— | 1 | 4-pyridyl |
| 219 | —C(S)—N(Me)— | 1 | 4-ethyl-3-pyridyl |
| 220 | —C(S)—N(Me)— | 1 | 2-thienyl |
| 221 | —C(S)—N(Me)— | 1 | 2-benzothienyl |
| 222 | —C(S)—N(Me)— | 1 | 3-chloro-2-thienyl |
| 223 | —C(S)—N(Me)— | 1 | 1-cyclohexyl |
| 224 | —C(S)—N(Me)— | 1 | 1-cyclopentyl |
| 225 | —C(S)—N(Me)— | 1 | 2-chloro-5-thiazolyl |
| 226 | —C(S)—N(Me)— | 1 | 3,5-dimethyl-4-isoxazolyl |
| 227 | —C(O)—NH—S(O)₂— | 1 | 2-bromo-phenyl |
| 228 | —C(O)—NH— | 2 | 2-[3-chloro-5-(trifluoromethyl)- |
| | | | 2-pyridyl]-1-ethyl |
| 229 | —C(O)—NH— | 2 | 3-[(2-trifluoromethyl)-phenyl]- |
| | | | 1,2,4-thiadiazol-5-yl |
| 230 | —C(O)—NH— | 2 | 3-[(2,3,5,6-tetrafluoro)-phenyl]- |
| | | | 1,2,4-thiadiazol-5-yl |
| 231 | —C(O)—NH— | 2 | 2-chloro-5-methoxy-phenyl |

For use in agriculture it is often advantageous to use compositions having a nematocidal activity containing, as active substance, one or more compounds having general formula (XVI), possibly also as a mixture of isomers.

Depending on the development phase of the parasite and the quantity applied, the compounds having formula (XVI) can act as nematostatic and/or nematocidal agents.

A third object of the present invention therefore relates to the use of the compounds having formula (XVI) as nematocides.

A fourth object of the present invention relates to an agronomic composition for the treatment and prevention of phytoparasitosis from nematodes comprising at least one compound having general formula (XVI) and optionally at least one carrier and/or at least one excipient suitable for application to plants and/or to the soil. The term carrier refers to a compound that can dissolve, dilute or disperse the active compound. The term excipient refers to a compound that can modify one or more properties of the composition, contributing to obtaining a certain technical effect such as a higher stability of the composition, a better applicability of the same to plants or to the soil and the like.

Compositions can be used that are in the form of dry powders, wettable powders, emulsifiable concentrates, microemulsions, pastes, granulates, solutions, suspensions, fumigants, etc.: the choice of the type of composition will depend on the specific use.

At least one carrier, i.e. at least one solvent or at least one diluent or mixtures thereof, can be present in the present composition. The diluent can be solid or liquid. The compositions are prepared according to known methods, for example by diluting or dissolving the active substance with a solvent and/or solid diluent medium, possibly in the presence of surfactants.

Kaolin, alumina, silica, talc, bentonite, plaster, quartz, dolomite, attapulgite, montmorillonite, diatomaceous earth, cellulose, starch, etc. can be used as solid inert diluents, or carriers.

Water or organic solvents such as aromatic hydrocarbons (xylols, mixtures of alkylbenzenes, etc.), aliphatic hydrocarbons (hexane, cyclohexane, etc.), halogenated aromatic hydrocarbons (chlorobenzene, etc.), alcohols (methanol, propanol, butanol, octanol, etc.), esters (isobutyl acetate, etc.), ketones (acetone, cyclohexanone, acetophenone, isophorone, ethylamylketone, etc.), or vegetable or mineral oils or their mixtures, etc. can be used as liquid inert diluents.

One or more excipients can be present in the present composition.

Wetting agents and emulsifiers of the non-ionic type (polyethoxylated alkylphenols, polyethoxylated fatty alcohols, etc.), of the anionic type (alkylbenzenesulfonates, alkylsulfonates, etc.), of the cationic type (quaternary salts of alkylammonium, etc.), can be used as surfactants.

Propellant gases such as butane, propane, halogenated hydrocarbons, nitrogen or carbon dioxide can be used as liquefied diluents or liquefied substances that gasify at ambient temperature and pressure.

Dispersants (e.g. lignin and its salts, cellulose derivatives, alginates, etc.), stabilizers (e.g. antioxidants, ultraviolet ray absorbents, etc.) can also be added. The compounds claimed in this patent application as such or formulated can be used in a mixture with other active principles such as, for example, herbicides, fungicides, bactericides, insecticides, acaricides, nematocides, fertilizers, biostimulants etc. to broaden the spectrum or prevent resistance.

In some cases, this results in a synergistic effect between the components that leads the mixture, for example, to exert a higher activity than that of the individual elements composing it.

Examples of fungicides that can be added to compositions containing one or more compounds having general formula (XVI) are the following: acibenzolar, ametoctradin, amisulbrom, ampropylfos, anilazine, azaconazole, azoxystrobin, benalaxyl, benalaxyl-M, benomyl, benthiavalicarb, bitertanol, bixafen, blasticidin-S, boscalid, bromuconazole, bupirimate, buthiobate, captafol, captan, carbendazim, carboxin, carpropamid, chinomethionat, chloroneb, chlorothalonil, chlozolinate, cyazofamid, cyflufenamid, cymoxanil, cyproconazole, cyprodinil, debacarb, dichlofluanid, dichlone, diclobutrazol, diclomezine, dicloran, diclocymet, diethofencarb, difenoconazole, diflumetorim, dimethirimol, dimethomorph, dimoxystrobin, diniconazole, dinocap, dipyrithione, ditalimfos, dithianon, dodemorph, dodine, edifenphos, epoxiconazole, etaconazole, ethaboxam, ethirimol, ethoxyquin, etridiazole, famoxadone, fenamidone, fenaminosulf, fenapanil, fenarimol, fenbuconazole, fenfuram, fenhexamid, fenoxanil, fenpiclonil, fenpropidin, fenpropimorph, fenpyrazamine, fentin, ferbam, ferimzone, fluazinam, fludioxonil, fluindapyr, flumetover, flumorph, fluopicolide, fluopyram, fluoroimide, fluotrimazole, fluoxastrobin, fluquinconazole, flusilazole, flusulfamide, flutianil, flutolanil, flutriafol, fluxapyroxad, folpet, fosetyl-aluminium, fuberidazole, furalaxyl, furametpyr, furconazole, furconazole-cis, guazatine, hexaconazole, hymexazol, hydroxyquinoline sulfate, imazalil, imibenconazole, iminoctadine, ipconazole, iprobenfos, iprodione, isoprothiolane, iprovalicarb, isopyrazam, isotianil, kasugamycin, kresoxim-methyl, mancopper, mancozeb, mandipropamid, maneb, mebenil, mepanipyrim, mepronil, meptyldinocap, metalaxyl, metalaxyl-M, metconazole, methfuroxam, metiram, metominostrobin, metrafenone, metsulfovax, myclobutanil, natamycin, nicobifen, nitrothal-isopropyl, nuarimol, ofurace, orysastrobin, oxadixyl, oxpoconazole, oxycarboxin, pefurazoate, penconazole, pencycuron, penflufen, pentachlorofenol and its salts, penthiopyrad, phthalide, picoxystrobin, piperalin, Bordeaux mixture, polyoxins, probenazole, prochloraz, procymidone, propamocarb, propiconazole, propineb, proquinazid, prothiocarb, prothioconazole, pyracarbolid, pyraclostrobin, pyrametostrobin, pyraoxystrobin, pyrazophos, pyribencarb, pyrifenox, pyrimethanil, pyriofenone, pyroquilon, pyroxyfur, quinacetol, quinazamid, quinconazole, quinoxyfen, quintozene, rabenzazole, copper hydroxide, copper oxychloride, copper (I) oxide, copper sulfate, sedaxane, silthiofam, simeconazole, spiroxamine, streptomycin, tebuconazole, tebufloquin, tetraconazole, thiabendazole, thiadifluor, thicyofen, thifluzamide, thiophanate, thiophanate-methyl, thiram, tiadinil, tioxymid, tolclofos-methyl, tolylfluanid, triadimefon, triadimenol, triarimol, triazbutil, triazoxide, tricyclazole, tridemorf, trifloxystrobin, triflumizole, triforine, triticonazole, uniconazole, uniconazole-P, validamycin, valifenalate, vinclozolin, zineb, ziram, sulfur, zoxamide.

Examples of herbicides that can be added to compositions containing one or more compounds having general formula (XVI) are the following: acetochlor, acifluorfen, aclonifen, AKH-7088, alachlor, alloxydim, ametryn, amicarbazone, amidosulfuron, amitrole, anilofos, asulam, atrazine, azafenidin, azimsulfuron, aziprotryne, BAY MKH 6561, beflubutamid, benazolin, benfluralin, benfuresate, bensulfuron, bensulide, bentazone, benzfendizone, benzobicyclon, benzofenap, benzthiazuron, bifenox, bilanafos, bispyribac-sodium, bromacil, bromobutide, bromofenoxim, bromoxynil, butachlor, butafenacil, butamifos, butenachlor, butralin, butroxydim, butylate, cafenstrole, carbetamide, carfentrazone-ethyl, chlomethoxyfen, chloramben, chlorbromuron, chlorbufam, chlorflurenol, chloridazon, chlorimuron, chlornitrofen, chlorotoluron, chloroxuron, chlorpropham, chlorsulfuron, chlorthal, chlorthiamid, cinidon ethyl, cinmethylin, cinosulfuron, clethodim, clodinafop, clomazone, clomeprop, clopyralid, cloransulam-methyl, cumyluron (JC-940), cyanazine, cycloate, cyclosulfamuron, cycloxydim, cyhalofop-butyl, 2,4-D, 2,4-DB, daimuron, dalapon, desmedipham, desmetryn, dicamba, dichlobenil, dichlorprop, dichlorprop-P, diclofop, diclosulam, diethatyl, difenoxuron, difenzoquat, diflufenican, diflufenzopyr, dimefuron, dimepiperate, dimethachlor, dimethametryn, dimethenamid, dinitramine, dinoseb, dinoseb acetate, dinoterb, diphenamid, dipropetryn, diquat, dithiopyr, 1-diuron, eglinazine, endothal, EPTC, esprocarb, ethalfluralin, ethametsulfuron-methyl, ethidimuron, ethiozin (SMY 1500), ethofumesate, ethoxyfen-ethyl (HC-252), ethoxysulfuron, etobenzanid (HW 52), fenoxaprop, fenoxaprop-P, fentrazamide, fenuron, flamprop, flamprop-M, flazasulfuron, florasulam, fluazifop, fluazifop-P, fluazolate (JV 485), flucarbazone-sodium, fluchloralin, flufenacet, flufenpyr ethyl, flumetsulam, flumiclorac-pentyl, flumioxazin, flumipropin, fluometuron, fluoroglycofen, fluoronitrofen, flupoxam, flupropanate, flupyrsulfuron, flurenol, fluridone, flurochloridone, fluroxypyr, flurtamone, fluthiacet-methyl, fomesafen, foramsulfuron, fosamine, furyloxyfen, glufosinate, glyphosate, halosulfuron-methyl, haloxyfop, haloxyfop-P-methyl, hexazinone, imazamethabenz, imazamox, imazapic, imazapyr, imazaquin, imazethapyr, imazosulfuron, indanofan, iodosulfuron, ioxynil, isopropalin, isoproturon, isouron, isoxaben, isoxachlortole, isoxaflutole, isoxapyrifop, KPP-421, lactofen, lenacil, linuron, LS830556, MCPA, MCPA-thioethyl, MCPB, mecoprop, mecoprop-P, mefenacet, mesosulfuron, mesotrione, metamitron, metazachlor, methabenzthiazuron, methazole, methoprotryne, methyldymron, metobenzuron, metobromuron, metolachlor, S-metolachlor, metosulam, metoxuron, metribuzin, metsulfuron, molinate, monalide, monolinuron, naproanilide, napropamide, naptalam, NC-330, neburon, nicosulfuron, nipyraclofen, norflurazon, orbencarb, oryzalin, oxadiargyl, oxadiazon, oxasulfuron, oxaziclomefone, oxyfluorfen, paraquat, pebulate, pendimethalin, penoxsulam, pentanochlor, pentoxazone, pethoxamid, phenmedipham, picloram, picolinafen, piperophos, pretilachlor, primisulfuron, prodiamine, profluazol, proglinazine, prometon, prometryne, propachlor, propanil, propaquizafop, propazine, propham, propisochlor, propyzamide, prosulfocarb, prosulfuron, pyraclonil, pyraflufen-ethyl, pyrazogyl (HSA-961), pyrazolynate, pyrazosulfuron, pyrazoxyfen, pyribenzoxim, pyributicarb, pyridafol, pyridate, pyriftalid, pyriminobac-methyl, pyrithiobac-sodium, pyroxasulfone quinclorac, quinmerac, quizalofop, quizalofop-P, rimsulfuron, sethoxydim, siduron, simazine, simetryn, sulcotrione, sulfentrazone, sulfometuron-methyl, sulfosulfuron, 2,3,6-TBA, TCA-sodium, tebutam, tebuthiuron, tepraloxydim, terbacil, terbumeton, terbuthyl-azine, terbutryn, thenylchlor, thiazafluron, thiazopyr, thidiazimin, thifensulfuron-methyl, thiobencarb, tiocarbazil, tioclorim, tralkoxydim, tri-allate, triasulfuron, triaziflam, tribenuron, triclopyr, trietazine, trifloxysulfuron, trifluralin, triflusulfuron-methyl, tritosulfuron, UBI-C4874, vernolate.

Examples of bactericides that can be added to compositions containing one or more compounds having general formula (XVI) are the following: bronopol, dichlorophen, nitrapyrina, nickel dimethyldithiocarbamate, kasugamycin, octhilinone, furancarboxylic acid, probenazole, streptomycin, tecloftalam, copper hydroxide, copper oxychloride, copper (I) oxide, copper sulfate, copper salicylate.

Examples of insecticides, acaricides, nematocides that can be added to the compositions containing one or more compounds having general formula (XVI) are the following:

abamectin, acetamiprid, acrinathrin, alphacypermethrin, alphamethrin, azadirachtin, Bacillus subtilis, Bacillus thuringiensis, Beauveria bassiana, betacyfluthrin, bifenazate, bifenthrin, buprofezin, chlorpyrifos, chlorpyrifos M, clofentezine, cyhalothrin, cyhexatin, cypermethrin, cyromazine, chloropicrin,clorantranilipide, clotianidin, deltamethrin, diflubenzuron, dimethoat, dazonet, difluoruro di solforile, dimethyldisulfide, emamectin, esfenvalerate, ethoprophos, etofenprox, etoxazole, fenamiphos, fenazaquin, fenoxycarb, fenpyroximate, fipronil, fluazinam, fluensulfone, flufenoxuron, fluvalinate, fosthiazate, formentanate, flonicamid, formet, viruses, hexythiazox, imidaclopridi, indoxacarb, lambda-cyhalothrin, lufenuron malathion, metaldehyde, methamidophos, Metharhizium spp, methiocarb, methomyl, methoxyfenozide, milbemectin, metaflumizone, metam sodium, metam potassium, oxamyl, Paecilomyces fumosoroseus, phosmet, pirimicarb, pirimiphos M, pyrethrum, pyridaben, pyriproxyfen, piperonyl butoxide, spinosad, spironesifen, spirotetramat, spinetoran, spirodiclofen, tau-fluvalinate, tebufenozide, tebufenpyrad, teflubenzuron, tefluthrin, thiacloprid, triflumuron, zeta-cypermethrin, (1R-cis)-[5-(phenylmethyl)-3-furanyl]-methyl-3-[(dihydro-2-oxo-3(2H)-furanylidene)methyl]-2,2-dimethyl-cyclopropanecarboxylate, (3-phenoxyphenyl)-methyl-2,2,3,3-tetramethyl-cyclopropane-carboxylate, 1-[(2-chloro-5-thiazolyl)methyl]-5-triazine 2-(1H)-imine, 2-(2-chloro-6-fluorophenyl)-4-[4-(1,1-dimethylethyl)phenyl]-4,5-dihydro-oxazole, 2-(acetyloxy)-3-dodecyl-1,4-naphthalenedione, 2-chloro-N-[[[4-(1-phenylethoxy)-phenyl]-amino]-carbonyl]-benzamide, 2-chloro-N-[[[4-(2,2-dichloro-1,1-difluoroethoxy)-phenyl]-amino]-carbonyl]-benzamide, 3-methylphenyl-propylcarbamate, 4-[4-(4-ethoxyphenyl)-4-methylpentyl]-1-fluoro-2-phenoxybenzene, 4-chloro-2-(1,1-dimethylethyl)-5-[[2-(2,6-dimethyl-4-phenoxyphenoxy)ethyl]thio]-3-(2H)-pyridazinone, 4-chloro-2-(2-chloro-2-methylpropyl)-5-[(6-iodo-3-pyridinyl)(2-chloro-2-methylpropyl)-5-[(6-iodo-3-pyridinyl)-methoxy]-3-(2H)pyridazinone, 4-chloro-5-[(6-chloro-3-pyridinyl)methoxy]-2-(3,4-dichlorophenyl)-3(2H)pyridazinone, Bacillus thuringiensis strain EG-2348, [2-benzoyl-1-(1,1-dimethylethyl)-hydrazinobenzoic acid, 2,2-dimethyl-3-(2,4-dichlorophenyl)-2-oxo-1-oxaspiro[4.5]-dec-3-en-4-yl butanoate, [3-[(6-chloro-3-pyridinyl)-methyl]-2-thiazolidinyli-dene]-cyanamide, dihydro-2-(nitromethylene)-2H-1,3-thiazine-3(4H)-carboxaldehyde, ethyl[2-[[1,6-dihydro-6-oxo-1-(phenylmethyl)-4-pyridazinyl]oxy]ethyl]-carbamate, N-(3,4,4-trifluoro-1-oxo-3-butenyl)-glycine, N-(4-chlorophenyl)-3-[4-(difluoromethoxy)-phenyl]-4,5-dihydro-4-phenyl-1H-pyrazole-1-carboxamide, N-[(2-chloro-5-thiazolyl)methyl]-N′-methyl-N″-nitro-guanidine, N-methyl-N′-(1-methyl-2-propenyl)-1,2-hydrazinedicarbo-thioamide, N-methyl-N′-2-propenyl-1,2-hydrazinedicarbo-thioamide, O,O-diethyl[2-(dipropylamino)-2-oxoethyl]-ethyl-phosphoroamidothioate, 2-(propan-2-yl)-5-(3,4,4-trifluorobut-3-ene-1-sulfonyl)-1,3,4-thiadiazole.

Examples of fertilizers and biostimulants that can be added to compositions containing one or more compounds having general formula (XVI) are the following:

mixtures of amino acids and/or oligopeptides of animal and/or vegetable origin, 4-thiazolidinecarboxylic acid, 4-acetylthiazolidine-carboxylic acid, ectoin, phytosterols.

The concentration of active substance in the above-mentioned compositions can vary within a wide range, depending on the active compound, the applications for which they are intended, the environmental conditions and the type of formulation adopted. In general, the concentration of active substance ranges from 0.1 to 99%, preferably ranging from 0.5 to 90%.

In the method and composition according to the invention, at least one active compound is preferably selected from the compounds having formula (I), (I-A), (I-B), (I-C), (I-D), (I-E) as previously defined.

Some examples are now provided which are intended to be descriptive and non-limiting of the present invention.

### Example 1

### Preparation of 3,4,4-trifluorobut-3-en-1-yl (1,3-dimethyl-2,6-dioxo-1,2,3,6-tetrahydro-7H-purin-7-yl) acetate (General Formula (Id), Compound 21)

A) Synthesis of tert-butyl (1,3-dimethyl-2,6-dioxo-1,2,3,6-tetrahydro-7H-purin-7-yl) acetate. 2 g (14.44 mmoles) of K₂CO₃ are added to a solution of 1,3-dimethyl-3,7-dihydro-1H-purin-2,6-dione (theophylline) (IV) (2 g, 11.11 mmoles) in DMF (20 mL), and the mixture is left under stirring for 20 minutes at room temperature. 3.46 g (17.77 mmoles) of tert-butyl bromoacetate are then added; the mixture is heated to 85° C. for 12 hours. At the end of the reaction, the solution is poured into a mixture of H₂O/AcOEt, the phases are separated, the organic phase is washed with water and subsequently with brine. The organic phase obtained is anhydrified with sodium sulfate, filtered and evaporated. The residue is subjected to trituration with heptane; 2.86 g of the desired compound are obtained as a white solid (yield=88%). LC-MS [M⁺H⁺]=295.

B) Synthesis of (1,3-dimethyl-2,6-dioxy-1,2,3,6-tetrahydro-7H-purin-7-yl)-acetic acid. 2.86 g of tert-butyl-(1,3-dimethyl-2,6-dioxy-1,2,3,6-tetrahydro-7H-purin-7-yl) acetate (XIV) are dissolved in 24 mL of a 1:1 mixture of CH₂Cl₂/TFA at room temperature. The mixture is left under stirring at this temperature until the starting product disappears. The reaction is then recovered by evaporating the solvent; the residue is triturated with Et₂O, a white solid is thus obtained (2.34 g, quantitative yield).

LC-MS [M⁺H⁺]=239.

C) Synthesis of 3,4,4-trifluorobut-3-en-1-yl (1,3-dimethyl-2,6-dioxy-1,2,3,6-tetrahydro-7H-purin-7-yl) acetate. 1 g of (1,3-dimethyl-2,6-dioxy-1,2,3,6-tetrahydro-7H-purin-7-yl) acetic acid (X) is dissolved in 10 mL of DMF, and 460 mg (5.5 mmoles) of NaHCO₃ are subsequently added. The suspension is stirred at room temperature for 20 minutes and 0.8 mL of 4-bromo-1,1,2-trifluoro-1-butene (6.72 mmoles) are then added. After 12 hours at room temperature, an additional 0.3 mL of 4-bromo-1,1,2-trifluoro-1-butene (III) and 177 mg of NaHCO₃ are added, and the mixture is then heated to 80° C. for 8 hours. The reaction mixture is diluted in H₂O/AcOEt, the phases are separated, the organic phase is washed with water and subsequently with brine. The organic phase obtained is anhydrified with sodium sulfate, filtered and evaporated. The residue is purified by trituration with heptane; 1.18 g of the desired product are obtained (yield 81%).

LC-MS [M⁺H⁺]=347.

### Example 2

### Preparation of 4,4-difluorobut-3-en-1-yl (1,3-dimethyl-2,6-dioxy-1,2,3,6-tetrahydro-7H-purin-7-yl) acetate (General Formula (Id), Compound 22)

300 mg (1.75 mmoles) of 4-bromo-1,1-difluorobut-1-ene (III) and 200 mg (1.51 mmoles) of K₂CO₃ are added to a solution of 280 mg (1.16 mmoles) of (1,3-dimethyl-2,6-dioxo-1,2,3,6-tetrahydro-7H-purin-7-yl) acetic acid (X) in 10 mL of DMF. The reaction mixture is stirred at room temperature for 72 hours. At the end of the reaction, the mixture is diluted in H₂O/CH₂Cl₂, the phases are separated and the organic phase washed with water (2×) and brine (1×). The organic phase is finally dried with sodium sulfate, filtered and evaporated. The residue is purified by trituration; 130 mg of the desired product are obtained (yield=35%).

LC-MS [M⁺H⁺]: 329.

### Example 3

### Preparation of 1,3-dimethyl-7-{2-[(3,4,4-trifluorobut-3-en-1-yl) sulfanyl]ethyl}-3,7-dihydro-1H-purin-2,6-dione (General Formula (Ic), Compound 7)

A) Synthesis of 7-(2-bromoethyl)-1,3-dimethyl-3,7-dihydro-1H-purin-2,6-dione. 730 mg (18.3 mmoles) of NaH (60% in mineral oil) are added at room temperature to a solution of 1,3-dimethyl-3,7-dihydro-1H-purin-2,6-dione (theophylline) (IV) (3 g, 16.65 mmoles) in DMF (55 mL). The mixture is left under stirring for about 10 minutes, and 7.2 mL of dibromoethane (V) (83.25 mmoles) are then added dropwise. The mixture is left under stirring at room temperature for 24 hours; the reaction is then recovered by abundantly diluting with EtOAc and washing the organic phase with H₂O (3×) and brine (1×). The organic phase is anhydrified, filtered and evaporated. A white solid is obtained after trituration with heptane (3.97 g, yield=83%).

LC-MS [M⁺H⁺]: 288.

B) Synthesis of 2-(1,3-dimethyl-2,6-dioxy-1,2,3,6-tetrahydro-7H-purin-7-yl)ethyl thiouronium bromide. 1.9 g of 7-(2-bromoethyl)-1,3-dimethyl-3,7-dihydro-1H-purin-2,6-dione (VI) (6.62 mmoles) are dissolved in 10 mL of anhydrous ethanol, and 500 mg of thiourea (6.62 mmoles) are then added. The reaction mixture is stirred under reflux. After 12 hours, the reaction is brought to room temperature, and subsequently diluted with 15 mL of Et₂O. The heterogeneous mixture is left under stirring for 10 minutes and then filtered; the solid is washed with Et₂O. 2.09 g of a white solid are obtained, which are directly used for the subsequent reaction. Yield=87%.

LC-MS [M⁺H⁺]: 283.

C) Synthesis of 1,3-dimethyl-7-{2-[(3,4,4-trifluorobut-3-en-1-yl) sulfanyl]ethyl}-3,7-dihydro-1H-purin-2,6-dione. 2.09 g of 2-(1,3-dimethyl-2,6-dioxy-1,2,3,6-tetrahydro-7H-purin-7-yl)ethyl thiouronium bromide (VII) are suspended in 6 mL of a 3 M solution of NaOH, and the mixture is left under stirring for 30 minutes at room temperature. An ethanol solution (3 mL) of 0.85 mL (7.41 mmoles) of 4-bromo-1,1,2-trifluoro-1-butene (III) is subsequently added dropwise to the reaction mixture; at the end of the addition, the mixture is heated under reflux for 2 hours. The reaction is then diluted in EtOAc and washed with water and brine. The organic phase is then dried with sodium sulfate, filtered and evaporated. 2.32 g of the desired compound are obtained as a yellow oil (Yield=90%). LC-MS [M⁺H⁺]: 349.

### Example 4

### Preparation of 1,3-dimethyl-7-[2-(3,4,4-trifluorobut-3-ene-1-sulfinyl)ethyl]-3,7-dihydro-1H-purin-2,6-dione (General Formula (Ia), Compound 8)

700 mg of m-chloroperbenzoic acid are added at 0° C. to a solution of 1 g of 1,3-dimethyl-7-{2-[(3,4,4-trifluorobut-3-en-1-yl)sulfanyl]ethyl}-3,7-dihydro-1H-purin-2,6-dione (general formula (Ic), compound 7) in 23 mL of chloroform. The mixture is stirred at room temperature. At the end of the transformation, the reaction is poured into a 5% solution of Na₂S₂O₅, and the mixture is left under stirring for 10 minutes. The phases are separated, the organic phase is washed with NaHCO₃ (2×), water and brine, and subsequently anhydrified with sodium sulfate, filtered and evaporated. A white solid is obtained (700 mg, Yield=70%) further purified by trituration in heptane.

LC-MS [M⁺H⁺]: 365.

### Example 5

### Preparation of 1,3-dimethyl-7-[2-(3,4,4-trifluorobut-3-ene-1-sulfonyl)ethyl]-3,7-dihydro-1H-purin-2,6-dione (General Formula (Ib), Compound 11)

2.23 g of m-chloroperbenzoic acid are added at 0° C. to a solution of 1.16 g of 1,3-dimethyl-7-{2-[(3,4,4-trifluorobut-3-en-1-yl) sulfanyl]ethyl}-3,7-dihydro-1H-purin-2,6-dione (general formula (Ic), compound 7) in 26 mL of chloroform. At the end of the addition, the reaction is stirred for 1 hour at 0° C. and subsequently at room temperature. At the end of the transformation, the reaction is poured into a 5% solution of Na₂S₂O₅, and the mixture is left under stirring for 10 minutes. The phases are separated, the organic phase is washed with NaHCO₃ (2×), water and brine, and subsequently anhydrified with sodium sulfate, filtered and evaporated. 390 mg of a white solid are obtained after purification by chromatography on silica (heptane/ethyl acetate gradient). (Yield=30%).

LC-MS [M⁺H⁺]: 380.

### Example 6

### 2-(1,3-dimethyl-2,6-dioxo-1,2,3,6-tetrahydro-7H-purin-7-yl)-N-(3,4,4-trifluorobut-3-en-1-yl)acetamide (General Formula (Ih), Compound 131)

0.63 g of (1,3-dimethyl-2,6-dioxy-1,2,3,6-tetrahydro-7H-purin-7-yl) acetic acid (X) (2.66 mmoles) are dissolved in a 1:1 mixture of CH₂Cl₂ and tBuOH (26 mL), and 1.03 g of EDC hydrochloride (6.65 mmoles) and 0.97 g of DMAP (7.98 mmoles) are subsequently added in sequence. The reaction mixture is then left under stirring under nitrogen at room temperature for 15 minutes; 0.5 g of 3,4,4-trifluorobuten-3-ylammonium chloride (4 mmol) are subsequently added. After 48 hours at this temperature, the reaction mixture is diluted with water and methylene chloride, the phases are separated, the aqueous phase is extracted with methylene chloride (2×); the combined organic phases are washed with water (1×) and brine (1×), dried with sodium sulfate, filtered and evaporated. The residue is triturated with a mixture of diethyl ether and heptane; 300 mg of the desired compound are obtained (yield 32%).

LC-MS [M⁺H⁺]=346.

### Example 7

### N-(2-bromobenzene-1-sulfonyl)-2-(1,3-dimethyl-2,6-dioxy-1,2,3,6-tetrahydro-7H-purin-7-yl)acetamide (general formula (If), compound 227)

1.63 g of EDC hydrochloride (10.50 mmoles) and 1.54 g of DMAP (12.6 mmoles) are added in sequence to a solution of 1 g of (1,3-dimethyl-2,6-dioxy-1,2,3,6-tetrahydro-7H-purin-7-yl)-acetic acid (X) (4.20 mmoles) in a 1:1 mixture of CH₂Cl₂ and tBuOH (42 mL). The reaction mixture is then left under stirring under nitrogen at room temperature for 15 minutes; 1.5 g of 2-bromobenzene-1-sulfonamide (6.3 mmoles) are subsequently added. After 12 hours at room temperature, the reaction mixture is diluted with water and methylene chloride, the phases are separated, the aqueous phase is extracted with methylene chloride (2×); the combined organic phases are washed with water (1×) and brine (1×), dried with sodium sulfate, filtered and evaporated. The residue is triturated with ethyl acetate; 1.42 g of the desired compound are obtained (74% yield).

LC-MS [M⁺H⁺]=457.

Operating analogously to the previous Examples, the compounds indicated in Table 4 were prepared.

TABLE 4

| Comp. | | | |
|---|---|---|---|
| Nr | A | n | E |
| 4 | —C(O)O— | 2 | —(CH₂)₂—CF═CF₂ |
| 67 | —C(O)—NH—S(O)₂— | 1 | —(CH₂)₂—CF═CF₂ |
| 74 | —C(O)—NH—S(O)₂— | 2 | —(CH₂)₂—CF═CF₂ |
| 98 | —C(O)—NH—S(O)₂— | 1 | 2-chloro-5-methoxy-phenyl |
| 134 | —C(O)—NH— | 1 | 3-[(2-trifluoromethyl)-phenyl]- |
| | | | 1,2,4-thiadiazol-5-yl |
| 135 | —C(O)—NH— | 1 | 3-[(2,3,5,6-tetrafluoro)-phenyl]- |
| | | | 1,2,4-thiadiazol-5-yl |
| 136 | —C(O)—NH— | 1 | 5-[(2-trifluoromethyl)-phenyl]- |
| | | | 1,3,4-thiadiazol-2-yl |
| 138 | —C(O)—NH— | 1 | 2-chloro-5-methoxy-phenyl |
| 228 | —C(O)—NH— | 2 | 2-[3-chloro-5-(trifluoromethyl)- |
| | | | 2-pyridyl]-1-ethyl |
| 229 | —C(O)—NH— | 2 | 5-[(2-trifluoromethyl)-phenyl]- |
| | | | 1,2,4-thiadiazol-3-yl |
| 230 | —C(O)—NH— | 2 | 3-[(2,3,5,6-tetrafluoro)-phenyl]- |
| | | | 1,2,4-thiadiazol-5-yl |
| 231 | —C(O)—NH— | 2 | 2-chloro-5-methoxy-phenyl |

Table 5 indicates the results of the LC-MS analyzes carried out on Compounds 4, 67, 74, 98, 134, 135, 136, 138, 228, 229, 230 and 231.

TABLE 5

| Comp. Nr | LC-MS: [M + H⁺] |
|---|---|
| 4 | 361 |
| 67 | 410 |
| 74 | 424 |
| 98 | 443 |
| 134 | 466 |
| 135 | 470 |
| 136 | 466 |
| 138 | 379 |
| 228 | 460 |
| 229 | 480 |
| 230 | 484 |
| 231 | 393 |

### Example 8

### Determination of the Nematocidal Activity Against Meloidogyne sp.

The tests aimed at testing the nematocidal activity of the product under examination are carried out using inocula taken from a cultivation of Meloidogyne sp. maintained on potted tomato and cucumber plants and grown in a greenhouse.

In order to carry out the experiments, portions of roots having a large number of galls and soil are taken from the infested pots, in which larvae are present starting from the second stage of age.

New pots having a diameter of 15 cm are half-filled with sterile earth. The portions of infested roots, previously cleaned, are placed on it to be able to correctly assess the degree of infestation and ensure that each pot contains the same nematic charge. 200-300 g of infested soil are then added, which are covered with a thin layer of sterile earth.

The treatment is carried out by pouring 100 ml of hydroacetonic solution onto the surface of the soil in which the product to be tested has been dissolved to obtain a concentration ranging from 70 to 100 ppm.

Tomato or cucumber seedlings at the stage of two or three actual leaves are transplanted in the pots thus prepared, after one or seven days from the application. Different cultivars of tomatoes (Microtom, Marmande) or cucumbers (Lungo della Cina) are used, having a different sensitivity to the parasite and different times of growth. In particular, for estimating the final production, a variety of ornamental tomatoes (cv Microtom) is used, whose seedlings are small in size and able to reach the maturity of the fruit in about two months in pots and under greenhouse conditions.

The containment capacity of the parasite is detected, 30 and 60 days after the transplantation, considering the development of the plant (whose fresh weight is detected) and the presence of galls on the roots.

This is estimated using both the infestation scale proposed by Bridge-Page (Tropical Pest Management 26 (3): 296-298, September 1980), according to which the value zero corresponds to 0% of root affected and the value 10 corresponds to 100% of infested root, and also considering the percentage of root mass affected.

Table 6 indicates the results relating to the effectiveness of Compounds Nr. 8, 11, 13, 21, and caffeine, on tomatoes, cv Marmande, at a dose of 4,000 g/hectare, carrying out the survey 30 days after the transplantation.

TABLE 6

| | GALL | | | |
|---|---|---|---|---|
| | INDEX | IN- | PLANT | PHYTO- |
| | (scale | FESTED | DEVELOPMENT | TOXICITY |
| COMP. Nr | 0-10) | ROOT % | (fresh weight g) | (%) |
| INFESTED | 7 | 70 | 13 | |
| BLANK | | | | |
| Caffeine | 7 | 70 | 12 | 20 |
| Comp. Nr. 8 | 5 | 50 | 17 | 0 |
| Comp. Nr. 11 | 4.5 | 45 | 16 | 0 |
| Comp. Nr. 13 | 4 | 40 | 17 | 0 |
| Comp. Nr. 21 | 2 | 20 | 20 | 0 |

As revealed by the data indicated in Table 6, all the compounds according to the invention show a gall index and a percentage of infested root lower than those of both infested and untreated plants (infested blank), and those treated with caffeine alone. In particular, compounds 11, 13 and 21 show a decisive reduction in parasitosis at the root level which is reflected in the improved well-being of the plants, whose weight appears to be significantly higher.

Finally, from the data tabulated herein, it can be observed how caffeine seems to be completely comparable with respect to infested and untreated plants (infested witness).

1. A compound having a formula (I): 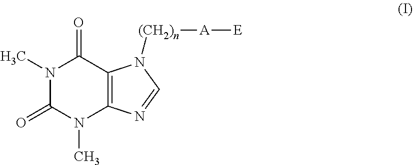
wherein:
n represents an integer ranging from 0 to 3;
A represents a group selected from: —S—, —S(═O)—, —S(═O)₂—, —C(═Y)—O—, —C(═Y)—NR₁—S(O)₂—;
Y represents an oxygen or sulfur atom;
R₁ represents a hydrogen atom, a C₁-C₆ alkyl group, a C₁-C₆ haloalkyl group, a C₂-C₆ alkenyl group, a C₂-C₆ haloalkenyl group, a C₃-C₈ cycloalkyl group, a C₄-C₉ cycloalkylalkyl group, a C(═Y)R₂ group, a C(═O)OR₂ group, a C(═O)NR₂R₃ group, an S(═O)ᵣR₂ group, an S(═O)₂NR₂R₃ group;
R₂ and R₃, the same as or different from each other, represent a hydrogen atom, a C₁-C₆ alkyl group, a C₁-C₆ haloalkyl group, a C₂-C₆ alkenyl group, a C₂-C₆ haloalkenyl group, a C₃-C₈ cycloalkyl group, a C₃-C₈ halocycloalkyl group, an aryl group, a benzyl group;
r represents an integer ranging from 0 to 2;
E represents a —(CH₂)ₘ—CX═CF₂ group or a group selected from a C₁-C₆ alkyl group, a C₁-C₆ haloalkyl group, a C₂-C₆ alkenyl group, a C₂-C₆ haloalkenyl group, a C₂-C₆ alkynyl group and a C₂-C₆ haloalkynyl group, optionally substituted by a C₁-C₆ alkoxyl group;
m represents an integer ranging from 1 to 6;
X represents a hydrogen or fluorine atom;
wherein when A represents a group selected from: —S—, —S(═O)—, —S(═O)₂—, —C(═Y)—O—, then E represents the group —(CH₂)ₘ—CX═CF₂.

2. The compound according to claim 1 having a formula selected from the group consisting of:
formula (I-A), wherein:
n is an integer ranging from 0 to 2,
R₁ represents a hydrogen atom or a C₁-C₆ alkyl group,
E represents a —(CH₂)ₘ—CX═CF₂ group or a group selected from a C₁-C₆ alkyl group and a C₁-C₆ haloalkyl group, optionally substituted by a C₁-C₆ alkoxyl group,
m represents an integer ranging from 1 to 5;
formula (I-B), wherein:
n is an integer selected from 1 and 2,
A represents a group selected from: —S(═O)—, —S(═O)₂—, —C(═O)—O—, —C(═O)—NR₁—S(═O)₂—,
R₁ represents a hydrogen atom or a C₁-C₆ alkyl group,
E represents a —(CH₂)ₘ—CX═CF₂ group or a C₁-C₆ alkyl group optionally substituted by a C₁-C₆ alkoxyl group,
m represents an integer ranging from 1 to 4;
formula (I-C), wherein:
n is an integer ranging from 1 to 2,
A represents a group selected from: —S(═O)—, —S(O)₂—, —C(═O)—O—, C(═O)—NR₁—S(═O)₂—,
R₁ represents a hydrogen atom or a C₁-C₆ alkyl group,
E represents a —(CH₂)ₘ—CX═CF₂ group,
m represents an integer ranging from 1 to 4;
formula (I-D), wherein:
E represents a (CH₂)ₘCX═CF₂ group, and
n is selected from 1 and 2;
and
formula (I-E), wherein:
E represents a (CH₂)ₘCX═CF₂ group,
m is 2,
n is selected from 1 and 2,
A represents a group selected from: —S(═O)—, —S(O)₂—, —C(═O)—O—, C(═O)—NR₁—S(═O)₂—.

3. The compound according to claim 1 of general formula (I), wherein n, A and E have the following meanings:

| Comp. Nr | A | n | E |
|---|---|---|---|
| 1 | —C(O)O— | 0 | —(CH₂)₂—CF═CF₂ |
| 2 | —C(O)O— | 0 | —(CH₂)₂—CH═CF₂ |
| 3 | —C(O)O— | 0 | —(CH₂)₄—CF═CF₂ |
| 4 | —C(O)O— | 2 | —(CH₂)₂—CF═CF₂ |
| 5 | —S— | 0 | —(CH₂)₂—CF═CF₂ |
| 6 | —S— | 0 | —(CH₂)₂—CH═CF₂ |
| 7 | —S— | 2 | —(CH₂)₂—CF═CF₂ |
| 8 | —S(O)— | 2 | —(CH₂)₂—CF═CF₂ |
| 9 | —S(O)— | 0 | —(CH₂)₂—CF═CF₂ |
| 10 | —S(O)— | 0 | —(CH₂)₂—CH═CF₂ |
| 11 | —S(O)₂— | 2 | —(CH₂)₂—CF═CF₂ |
| 12 | —S(O)— | 0 | —(CH₂)₄—CH═CF₂ |
| 13 | —S(O)₂— | 0 | —(CH₂)₂—CF═CF₂ |
| 14 | —S(O)₂— | 0 | —(CH₂)₂—CH═CF₂ |
| 15 | —S(O)₂— | 0 | —(CH₂)₄—CF═CF₂ |
| 16 | —S(O)₂— | 0 | —(CH₂)₄—CH═CF₂ |
| 17 | —C(S)O— | 0 | —(CH₂)₂—CF═CF₂ |
| 18 | —C(S)O— | 0 | —(CH₂)₂—CH═CF₂ |
| 19 | —C(S)O— | 0 | —(CH₂)₄—CF═CF₂ |
| 20 | —C(S)O— | 0 | —(CH₂)₄—CH═CF₂ |
| 21 | —C(O)O— | 1 | —(CH₂)₂—CF═CF₂ |
| 22 | —C(O)O— | 1 | —(CH₂)₂—CH═CF₂ |
| 23 | —C(O)O— | 1 | —(CH₂)₄—CF═CF₂ |
| 24 | —C(O)O— | 1 | —(CH₂)₄—CH═CF₂ |
| 25 | —S— | 1 | —(CH₂)₂—CF═CF₂ |
| 26 | —S— | 1 | —(CH₂)₂—CH═CF₂ |
| 27 | —S— | 1 | —(CH₂)₄—CF═CF₂ |
| 28 | —S— | 1 | —(CH₂)₄—CH═CF₂ |
| 29 | —S(O)— | 1 | —(CH₂)₂—CF═CF₂ |
| 30 | —S(O)— | 1 | —(CH₂)₂—CH═CF₂ |
| 31 | —S(O)— | 1 | —(CH₂)₄—CF═CF₂ |
| 32 | —S(O)— | 1 | —(CH₂)₄—CH═CF₂ |
| 33 | —S(O)₂— | 1 | —(CH₂)₂—CF═CF₂ |
| 34 | —S(O)₂— | 1 | —(CH₂)₂—CH═CF₂ |
| 35 | —S(O)₂— | 1 | —(CH₂)₄—CF═CF₂ |
| 36 | —S(O)₂— | 1 | —(CH₂)₄—CH═CF₂ |
| 37 | —C(S)O— | 1 | —(CH₂)₂—CF═CF₂ |
| 38 | —C(S)O— | 1 | —(CH₂)₂—CH═CF₂ |
| 39 | —C(S)O— | 1 | —(CH₂)₄—CF═CF₂ |
| 40 | —C(S)O— | 1 | —(CH₂)₄—CH═CF₂ |
| 41 | —C(O)—NH—S(O)₂— | 0 | —(CH₂)₂—CF═CF₂ |
| 42 | —C(O)—NH—S(O)₂— | 0 | —(CH₂)₂—CH═CF₂ |
| 43 | —C(O)—NH—S(O)₂— | 0 | 2-propyl |
| 44 | —C(O)—NH—S(O)₂— | 0 | 2-methyl-1-propyl |
| 45 | —C(O)—NH—S(O)₂— | 0 | 2-methoxy-1-ethyl |
| 46 | —C(O)—NH—S(O)₂— | 0 | 2-trifluoro-1-ethyl |
| 47 | —C(O)—NH—S(O)₂— | 0 | 3-bromo-1-butyl |
| 48 | —C(O)—NH—S(O)₂— | 1 | 2-propyl |
| 49 | —C(O)—NH—S(O)₂— | 1 | 2-methyl-1-propyl |
| 50 | —C(O)—NH—S(O)₂— | 1 | 2-methoxy-1-ethyl |
| 51 | —C(O)—NH—S(O)₂— | 1 | 2-trifluoro-1-ethyl |
| 52 | —C(O)—NH—S(O)₂— | 1 | 3-bromo-1-butyl |
| 53 | —C(O)—N(Me)—S(O)₂— | 0 | —(CH₂)₂—CF═CF₂ |
| 54 | —C(O)—N(Me)—S(O)₂— | 0 | —(CH₂)₂—CH═CF₂ |
| 55 | —C(O)—N(Me)—S(O)₂— | 0 | 2-propyl |
| 56 | —C(O)—N(Me)—S(O)₂— | 0 | 2-trifluoro-1-ethyl |
| 57 | —C(O)—N(Me)—S(O)₂— | 0 | 2-methyl-1-propyl |
| 58 | —C(O)—N(Me)—S(O)₂— | 0 | 2-methoxy-1-ethyl |
| 59 | —C(O)—N(Me)—S(O)₂— | 0 | 3-bromo-1-butyl |
| 60 | —C(O)—N(Me)—S(O)₂— | 1 | —(CH₂)₂—CF═CF₂ |
| 61 | —C(O)—N(Me)—S(O)₂— | 1 | —(CH₂)₂—CH═CF₂ |
| 62 | —C(O)—N(Me)—S(O)₂— | 1 | 2-propyl |
| 63 | —C(O)—N(Me)—S(O)₂— | 1 | 2-trifluoro-1-ethyl |
| 64 | —C(O)—N(Me)—S(O)₂— | 1 | 2-methyl-1-propyl |
| 65 | —C(O)—N(Me)—S(O)₂— | 1 | 2-methoxy-1-ethyl |
| 66 | —C(O)—N(Me)—S(O)₂— | 1 | 3-bromo-1-butyl |
| 67 | —C(O)—NH—S(O)₂— | 1 | —(CH₂)₂—CF═CF₂ |
| 68 | —C(O)—NH—S(O)₂— | 1 | —(CH₂)₂—CH═CF₂ |
| 69 | —C(O)—NH—S(O)₂— | 2 | 2-propyl |
| 70 | —C(O)—NH—S(O)₂— | 2 | 2-trifluoro-1-ethyl |
| 71 | —C(O)—NH—S(O)₂— | 2 | 2-methyl-1-propyl |
| 72 | —C(O)—NH—S(O)₂— | 2 | 2-methoxy-1-ethyl |
| 73 | —C(O)—NH—S(O)₂— | 2 | 3-bromo-1-butyl |
| 74 | —C(O)—NH—S(O)₂— | 2 | —(CH₂)₂—CF═CF₂ |
| 75 | —C(O)—NH—S(O)₂— | 2 | —(CH₂)₂—CH═CF₂ |
| 76 | —C(S)—NH—S(O)₂— | 1 | —(CH₂)₂—CF═CF₂ |
| 77 | —C(S)—NH—S(O)₂— | 1 | —(CH₂)₂—CH═CF₂ |
| 78 | —C(S)—NH—S(O)₂— | 1 | 2-propyl |
| 79 | —C(S)—NH—S(O)₂— | 1 | 2-trifluoro-1-ethyl |
| 80 | —C(S)—NH—S(O)₂— | 1 | 2-methyl-1-propyl |
| 81 | —C(S)—NH—S(O)₂— | 1 | 2-methoxy-1-ethyl |
| 82 | —C(S)—N(Me)—S(O)₂— | 1 | —(CH₂)₂—CF═CF₂ |
| 83 | —C(S)—N(Me)—S(O)₂— | 1 | —(CH₂)₂—CH═CF₂ |
| 84 | —C(S)—N(Me)—S(O)₂— | 1 | 2-propyl |
| 85 | —C(S)—N(Me)—S(O)₂— | 1 | 2-trifluoro-1-ethyl |
| 86 | —C(S)—N(Me)—S(O)₂— | 1 | 2-methyl-1-propyl |
| 87 | —C(S)—N(Me)—S(O)₂— | 1 | 2-methoxy-1-ethyl |
| 88 | —C(S)—NH—S(O)₂— | 2 | —(CH₂)₂—CF═CF₂ |
| 89 | —C(S)—NH—S(O)₂— | 2 | —(CH₂)₂—CH═CF₂ |
| 90 | —C(S)—NH—S(O)₂— | 2 | 2-propyl |
| 91 | —C(S)—NH—S(O)₂— | 2 | 2-trifluoro-1-ethyl |
| 92 | —C(S)—NH—S(O)₂— | 2 | 2-methyl-1-propyl |
| 93 | —C(S)—NH—S(O)₂— | 2 | 2-methoxy-1-ethyl |
| 94 | —C(S)—N(Me)—S(O)₂— | 2 | —(CH₂)₂—CF═CF₂ |
| 95 | —C(S)—N(Me)—S(O)₂— | 2 | —(CH₂)₂—CH═CF₂ |
| 96 | —C(S)—N(Me)—S(O)₂— | 2 | 2-propyl |
| 97 | —C(S)—N(Me)—S(O)₂— | 2 | 2-trifluoro-1-ethyl |

4. A method for controlling nematodes in cultivated areas comprising applying to the plant and/or soil; at least one compound
of formula (I) according to claim 1.

5. The method according to claim 4 wherein
the C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₃-C₈ cycloalkyl, C₃-C₈ halocycloalkyl, C₄-C₉ cycloalkylalkyl, aryl, naphthyl, benzyl, arylalkylene, heterocyclic or heterocyclylalkylene groups are non-substituted or substituted by one or more Q group(s) selected from halogen, C₁-C₆-alkyl, C₁-C₆-haloalkyl, C₃-C₆-cycloalkyl, C₄-C₉-cycloalkylalkyl, C₃-C₆-halocycloalkyl, C₁-C₆-alkoxy, C₁-C₆-haloalkoxy, C₁-C₆-thioalkoxy, C₁-C₆-thiohaloalkoxy, C₁-C₆-alkylsulfinyl, C₁-C₆-alkylsulfonyl, C₁-C₆-alkoxycarbonyl, C₃-C₆-cycloalkoxycarbonyl, amino, N—C₁-C₆-alkylamino, N,N—C₂-C₁₂-dialkylamino, N—C₁-C₆-alkoxycarbonylamino, N—C₃-C₆-cycloalkylamino, N,N—C₆-C₁₂-dicycloalkylamino, N—C₃-C₆-cycloalkoxycarbonylamino, C₁-C₆-alkylaminocarbonyl, C₃-C₆-cycloalkylaminocarbonyl, a NR₂R₃CONR₂— group; formyl, C₁-C₆-alkylcarbonyl, carboxyl, cyano, an optionally substituted aryl, a benzyl, a heterocyclic group, optionally substituted, penta- or hexa-atomic, aromatic or non-aromatic, also benzocondensed or heterobicyclic, containing at least one heteroatom selected from oxygen, sulfur, nitrogen, optionally oxidized to N-oxide, a (C₁-C₆)-alkyl-heterocyclic group, optionally substituted, penta- or hexa-atomic, also benzocondensed or heterobicyclic, containing at least one hetero atom selected from oxygen, sulfur, and nitrogen, optionally oxidized to N-oxide.

6. The method according to claim 4, wherein said nematodes are selected from the group consisting of Pratylenchus spp, Globodera spp, Heterodera spp, Meloidogyne spp, Aphelenchoides spp, Radopholus similis, Ditylenchus dipsaci, Tylenchulus semipenetrans, Longidorus spp, Xiphinema spp, Trichodorus spp, Bursaphelenchus spp. and Belonolaimus spp.

7. The method according to claim 4, wherein said compound is applied in the cultivated area at a dose ranging from 100 g to 10,000 g per hectare.

8. Use of a compound of formula (I) according to claim 1 as a nematostatic or nematocidal agent.

9. An agronomic composition for the treatment and prevention of phytoparasitosis from nematodes comprising at least one compound of formula (I) according to claim 1 and at least one carrier and/or at least one excipient suitable for application to plants and/or soil.

10. The agronomic composition according to claim 9, further comprising at least one second active compound selected from herbicides, fungicides, bactericides, insecticides, acaricides, nematocides, fertilizers and biostimulants.

11. The agronomic composition according to claim 9, wherein said compound is present at a concentration ranging from 0.1 to 99 by weight with respect to the total weight of the composition.

12. A compound having a general formula (XVI), 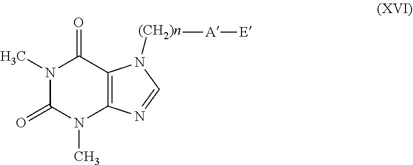
wherein:
n represents an integer ranging from 0 to 3;
A′ represents a group selected from: —S—, —S(O)—, —S(O)₂—, —C(═Y)—O—, —C(═Y)—NR₁—, —C(═Y)—NR₁—S(O)₂—;
Y represents an oxygen or sulfur atom;
R₁ represents a hydrogen atom, a C₁-C₆ alkyl group, a C₁-C₆ haloalkyl group, a C₂-C₆ alkenyl group, a C₂-C₆ haloalkenyl group, a C₃-C₈ cycloalkyl group, a C₄-C₉ cycloalkylalkyl group, a C(═Y)R₂ group, a C(═O)OR₂ group, a C(═O)NR₂R₃ group, an S(═O)ᵣR₂ group, an S(═O)₂NR₂R₃ group;
R₂ and R₃, the same as or different from each other, represent a hydrogen atom, a C₁-C₆ alkyl group, a C₁-C₆ haloalkyl group, a C₂-C₆ alkenyl group, a C₂-C₆ haloalkenyl group, a C₃-C₈ cycloalkyl group, a C₃-C₈ halocycloalkyl group, an aryl group, a benzyl group;
r represents an integer ranging from 0 to 2;
E′ represents a —(CH₂)ₘ—CX═CF₂ group, or an optionally substituted group selected from a C₁-C₆ alkyl, a C₁-C₆ haloalkyl group, a C₂-C₆ alkenyl group, a C₂-C₆ haloalkenyl group, a C₂-C₆ alkynyl group, a C₂-C₆ haloalkynyl group, a C₃-C₈ cycloalkyl group, a C₃-C₈ halocycloalkyl group, a C₄-C₉ cycloalkylalkyl group, an aryl group, a naphthyl group, a C₇-C₁₄ arylalkylene group, a heterocyclic group, penta- or hexa-atomic, aromatic or non-aromatic, also benzocondensed or heterobicyclic, containing at least one hetero atom selected from oxygen, sulfur, nitrogen, optionally oxidized to N-oxide, or a C₃-C₉ heterocyclylalkylene group wherein the heterocyclic group is as defined above;
m represents an integer ranging from 1 to 6;
X represents a hydrogen or fluorine atom;
wherein when A′ represents a group selected from: —S—, —S(═O)—, —S(═O)₂—, —C(═Y)—O—, then E′ represents the group —(CH₂)ₘ—CX═CF₂.

13. A method for controlling nematodes in cultivated areas by applying to the plant and/or soil, at least one compound of formula (XVI) according to claim 12.

14. The method according to claim 13, wherein said compound is applied in the cultivated area at a dose ranging from 100 g to 10,000 g per hectare.

15. The method according to claim 13 wherein:
the C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₃-C₈ cycloalkyl, C₃-C₈ halocycloalkyl, C₄-C₉ cycloalkylalkyl, aryl, naphthyl, benzyl, arylalkylene, heterocyclic or heterocyclylalkylene groups are non-substituted or substituted by one or more Q group(s) selected from halogen, C₁-C₆-alkyl, C₁-C₆-haloalkyl, C₃-C₆-cycloalkyl, C₄-C₉-cycloalkylalkyl, C₃-C₆-halocycloalkyl, C₁-C₆-alkoxy, C₁-C₆-haloalkoxy, C₁-C₆-thioalkoxy, C₁-C₆-thiohaloalkoxy, C₁-C₆-alkylsulfinyl, C₁-C₆-alkylsulfonyl, C₁-C₆-alkoxycarbonyl, C₃-C₆-cycloalkoxycarbonyl, amino, N—C₁-C₆-alkylamino, N,N—C₂-C₁₂-dialkylamino, N—C₁-C₆-alkoxycarbonylamino, N—C₃-C₆-cycloalkylamino, N,N—C₆-C₁₂-dicycloalkylamino, N—C₃-C₆-cycloalkoxycarbonylamino, C₁-C₆-alkylaminocarbonyl, C₃-C₆-cycloalkylaminocarbonyl, a NR₂R₃CONR₂— group; formyl, C₁-C₆-alkylcarbonyl, carboxyl, cyano, an optionally substituted aryl, a benzyl, a heterocyclic group, optionally substituted, penta- or hexa-atomic, aromatic or non-aromatic, also benzocondensed or heterobicyclic, containing at least one heteroatom selected from oxygen, sulfur, nitrogen, optionally oxidized to N-oxide, a (C₁-C₆)-alkyl-heterocyclic group, optionally substituted, penta- or hexa-atomic, also benzocondensed or heterobicyclic, containing at least one hetero atom selected from oxygen, sulfur, and nitrogen, optionally oxidized to N-oxide.

16. The method according to claim 13, wherein said nematodes are selected from the group consisting of Pratylenchus spp, Globodera spp, Heterodera spp, Meloidogyne spp, Aphelenchoides spp, Radopholus similis, Ditylenchus dipsaci, Tylenchulus semipenetrans, Longidorus spp, Xiphinema spp, Trichodorus spp, Bursaphelenchus spp. and Belonolaimus spp.

17. Use of a compound having formula (XVI) according to claim 12 as a nematostatic or nematocidal agent.

18. An agronomic composition for the treatment and prevention of phytoparasitosis from nematodes comprising at least one compound having formula (XVI) according to claim 12 and at least one carrier and/or at least one excipient suitable for application to plants and/or soil.

19. The agronomic composition according to claim 17, further comprising at least one second active compound selected from herbicides, fungicides, bactericides, insecticides, acaricides, nematocides, fertilizers and biostimulants.

20. The agronomic composition according to claim 17, wherein said compound is present at a concentration ranging from 0.1 to 99 by weight with respect to the total weight of the composition.
